SEQUENCE LISTING (21 sequences)

SEQ ID NO: 1 - protein, 434 aa, Neisseria meningitidis group B
  SITE 1..434: note = Polypeptide sequence of a MenB antigen 936-741
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 644 aa, Neisseria meningitidis (group B)
  SITE 1..644: note = Polypeptide sequence of a MenB antigen 287-953
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 3 - protein, 350 aa, Neisseria meningitidis (group B)
  SITE 1..350: note = Polypeptide sequence of a MenB antigen 961c
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 4 - protein, 390 aa, Neisseria meningitidis (group B)
  SITE 1..390: note = Polypeptide sequence of PorA (Neisseria meningitidis strain NZ98/254)
MRKKLTALVLSALPLAAVADVSLYGEIKAGVEGRNYQLQLTEAQAANGGASGQVKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQVLGIFKRHDDMPVSVRYDSPDFSGFSGSVQFVPIQNSKSAYTPAHVVVNNKVATHVPAVVGKPGSDVYYAGLNYKNGGFAGNYAFKYARHANVGRNAFELFLIGSATSDQAKGTDPLKNHQVHRLTGGYEEGGLNLALAAQLDLSENADKTKNSTTEIAATASYRFGNAVPRISYAHGFDLIERGKKGENTSYDQIIAGVDYDFSKRTSAIVSGAWLKRNTGIGNYTQINAASVGLRHKF

SEQ ID NO: 5 - protein, 386 aa, Gallus gallus
  SITE 1..386: note = Polypeptide sequence of chicken ovalbumin
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 6 - protein, 214 aa, Bos taurus
  SITE 1..214: note = Polypeptide sequence of bovine alpha-s1 casein
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 7 - protein, 217 aa, Gallus gallus
  SITE 1..217: note = Polypeptide sequence of fosvitin
AEFGTEPDAKTSSSSSSASSTATSSSSSSASSPNRKKPMDEEENDQVKQARNKDASSSSRSSKSSNSSKRSSSKSSNSSKRSSSSSSSSSSSSRSSSSSSSSSSNSKSSSSSSKSSSSSSRSRSSSKSSSSSSSSSSSSSSKSSSSRSSSSSSKSSSHHSHSHHSGHLNGSSSSSSSSRSVSHHSHEHHSGHLEDDSSSSSSSSVLSKIWGRHEIYQ

SEQ ID NO: 8 - protein, 708 aa, Bos taurus
  SITE 1..708: note = Polypeptide sequence of bovine lactotransferrin
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 9 - protein, 248 aa, Neisseria meningitidis
  SITE 1..248: note = Polypeptide sequence of a MenB antigen fHbp v1
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 10 - protein, 248 aa, Neisseria meningitidis
  SITE 1..248: note = Polypeptide sequence of the MenB antigen fHbp v1.13
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDEKGSYSLGIFGGQAQEVAGSAEVETANGIHHIGLAAKQ

SEQ ID NO: 11 - protein, 248 aa, Neisseria meningitidis
  SITE 1..248: note = Polypeptide sequence of the MenB antigen fHbp v1.13_E211A/S216R
VAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYRLGIFGGQAQEVAGSAEVETANGIHHIGLAAKQ

SEQ ID NO: 12 - protein, 251 aa, Neisseria meningitidis
  SITE 1..251: note = Polypeptide sequence of the MenB antigen fHbp v1.15
VAADIGAGLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGEHTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 13 - protein, 250 aa, Neisseria meningitidis
  SITE 1..250: note = Polypeptide sequence of the MenB antigen fHbp v1.55
VAADIGTGLADALTAPLDHKDKGLKSLTLEDSISQNGTLTLSAQGAEKTFKVGDKDNSLNTGKLKNDKISRFDFVQKIEVDGQTITLASGEFQIYKQDHSAVVALQIEKINNPDKIDSLINQRSFLVSGLGGEHTAFNQLPSGKAEYHGKAFSSDDAGGKLTYTIDFAAKQGHGKIEHLKTPEQNVELASAELKADEKSHAVILGDTRYGSEEKGTYHLALFGDRAQEIAGSATVKIREKVHEIGIAGKQ

SEQ ID NO: 14 - protein, 247 aa, Neisseria meningitidis
  SITE 1..247: note = Polypeptide sequence of a MenB antigen fHbp v2
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 15 - protein, 247 aa, Neisseria meningitidis
  SITE 1..247: note = Polypeptide sequence of a MenB antigen fHbp v2_S32V/L123R
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 16 - protein, 250 aa, Neisseria meningitidis
  SITE 1..250: note = Polypeptide sequence of a MenB antigen fHbp v3
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 17 - protein, 250 aa, Neisseria meningitidis
  SITE 1..250: note = Polypeptide sequence of a MenB antigen fHbp v3_S32V/L126R
VAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 18 - protein, 248 aa, Neisseria meningitidis
  SITE 1..248: note = Polypeptide sequence of the MenB antigen fHbp v3.45
VTADIGTGLADALTAPLDHKDKGLKSLTLEDSISQNGTLTLSAQGAEKTYGNGDSLNTGKLKNDKVSRFDFIRQIEVDGQLITLESGEFQVYKQSHSALTALQTEQEQDPEHSEKMVAKRRFRIGDIAGEHTSFDKLPKDVMATYRGTAFGSDDAGGKLTYTIDFAAKQGHGKIEHLKSPELNVDLAVAYIKPDEKHHAVISGSVLYNQDEKGSYSLGIFGEKAQEVAGSAEVETANGIHHIGLAAKQ

SEQ ID NO: 19 - protein, 757 aa, synthetic construct
  SITE 1..757: note = Polypeptide sequence of a MenB antigen fHbp v2-v3-v1#1
VAADIGAGLADALTAPLDHKDKSLQSLTLDQVVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 20 - protein, 755 aa, synthetic construct
  SITE 1..755: note = Polypeptide sequence of a MenB antigen fHbp v2-v3-v1#2
VAADIGAGLADALTAPLDHKDKSLQSLTLDQWRKNEKLKLAAQGAEKTYGNGDSLNTGKLKNDKVSRFDFIRQIEVDGQLITLESGEFQIYKQDHSAWALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAVVALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVSKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 21 - protein, 755 aa, synthetic construct
  SITE 1..755: note = Polypeptide sequence of a MenB antigen fHbp v2-v3-v1#3 (fHbp 23S_1.13_E211A/E232A)
VAADIGAGLADALTAPLDHKDKSLQSLTLDQWRKNEKLKLAAQGAEKTYGNGDSLNTGKLKNDKVSRFDFIRQIEVDGQPITPESGEFQIYKQDHSAVVAPQIEKINNPDKIDSPINQRSFRVSGPGGEHTAFNQPPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQGSGGGGVAADIGTGLADALTAPLDHKDKGLKSLTLEDVIPQNGTLTLSAQGAEKTFKAGDKDNSLNTGKLKNDKISRFDFVQKIEVDGQTITLASGEFQIYKQNHSAWALQIEKINNPDKTDSLINQRSFRVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQGSGGGGVAADIGAGLADALTAPLDHKDKGLQSLTLDQSVRKNEKLKLAAQGAEKTYGNGDSLNTGKLKNDKVSRFDFIRQIEVDGKLITLESGEFQVYKQSHSALTALQTEQVQDSEDSGKMVAKRQFRIGDIAGEHTSFDKLPKGGSATYRGTAFGSDDAGGKLTYTIDFAAKQGHGKIEHLKSPELNVELATAYIKPDEKRHAVISGSVLYNQDAKGSYSLGIFGGQAQEVAGSAAVETANGIHHIGLAAKQ

(19) United States
(12) Patent Application Publication
(10) Pub. No.: US 2025/0244344 A1
(43) Pub. Date: Jul. 31, 2025

(54) METHODS TO MEASURE THE CONCENTRATION OF ALUMINIUM HYDROXIDE IN MENB VACCINES

(71) Applicant: GLAXOSMITHKLINE BIOLOGICALS SA, Rixensart (BE)

(72) Inventors: Lorenzo DI MEOLA, Siena (IT); Agnese MARCELLI, Siena (IT); Malte MEPPEN, Siena (IT); Daniela PASQUI, Siena (IT); Daniela STRANGES, Siena (IT)

(73) Assignee: GLAXOSMITHKLINE BIOLOGICALS SA, Rixensart (BE)

(21) Appl. No.: 18/702,103

(22) PCT Filed: Oct. 20, 2022
(86) PCT No.: PCT/EP2022/079254

§ 371 Date: Apr. 17, 2024

(30) Foreign Application Priority Data
Oct. 21, 2021 (GB) 2115151.9

(51) Int. Cl. G01N 33/84

(52) U.S. Cl. CPC G01N 33/84; G01N 2333/22

(57) ABSTRACT

The present invention relates inter alia to methods of measuring the concentration of aluminium hydroxide in MenB vaccines.

### TECHNICAL FIELD

The present invention relates to measuring the concentration of aluminium hydroxide in MenB vaccines.

### BACKGROUND

Certain N. meningitidis serogroup B strain (MenB) vaccines comprise MenB antigens adsorbed to aluminium hydroxide (Al(OH)₃) adjuvant. Often these vaccines are produced in batches and in the form of multiple small containers, such as pre-filled syringes (PFS). During production, an important part of the quality control (QC) process is to continuously check that test samples of the vaccine comprise a consistent and acceptable quantity of aluminium hydroxide. Current methods used in QC for measuring aluminium hydroxide content involve complex procedures for test sample preparation and handling.

There exists a need to provide more efficient methods of measuring aluminium hydroxide content in test samples.

In general, aluminium adjuvants are composed of nanoscale primary particles. The aluminium hydroxide nanoparticles are elongate, approximately 4×2×10 nm in size. These nanoparticles form loosely connected porous aggregates that vary in size from about 1 to about 20 μm.

The aluminium hydroxide at the surface of the particles is coordinated with amphoteric hydroxyls that can accept or donate a proton depending on the pH of the solution. As a result, the aluminium hydroxide adjuvant has a pH-dependent surface charge. The point of zero charge or isoelectric point (iep) of aluminium hydroxide is a pH of 11.4 giving aluminium hydroxide a positive surface charge at neutral pH. Aluminium has a high affinity for phosphate, which can replace surface hydroxyls through ligand exchange. Aluminium has an even higher affinity for fluoride, moderate affinity for sulfate and low affinity for other anions such as chloride and nitrate. The adsorptive capacity of aluminium hydroxide surface adjuvant is therefore dependent on the composition of the buffers in which it is used.

Antigen adsorption to adjuvants occurs via hydrophobic, electrostatic, and ligand exchange mechanisms, among others. Purified or chemically synthesized antigens are large complex structures composed of proteins made up of a diverse array of amino acids, sometimes conjugated with oligo- or polysaccharide chains and lipids. This diversity makes it difficult to predict the adsorptive behaviour of proteins on aluminium adjuvants. Proteins tend to adsorb to solid surfaces and adsorption is generally highest when the pH approaches the iep of the protein. Electrostatic interactions occur when the protein and adjuvant have opposite charges and represent the major mechanism for adsorption of antigens to aluminium adjuvants. At neutral pH, aluminium hydroxide has a positive surface charge. The iep of protein antigens can be used as a starting point to determine if it is likely to undergo electrostatic adsorption to aluminium hydroxide. The strongest interaction between aluminium adjuvants and antigens is driven by the mechanism of ligand exchange. As mentioned above, aluminium has a high affinity for phosphate, and phosphate can exchange for hydroxyl groups at the surface of aluminium adjuvants. The presence and exposure of terminal phosphate groups in some antigens allows binding through ligand exchange. Binding of such antigens to aluminium adjuvants via ligand exchange can even overcome an electrostatic repulsion. Ligand exchange occurs with both types of aluminium adjuvants (aluminium hydroxide and aluminium phosphate), but it is stronger in aluminium hydroxide because this adjuvant has more surface hydroxyls available than aluminium phosphate.

Besides characterization of antigens adsorbed onto aluminium salts, it is also important to understand the effects of antigens once adsorbed to aluminium hydroxide. Due to the heterogeneous nature of aluminium adjuvant, the aluminium hydroxide particle size may be dependent by antigen adsorption on its surface resulting in different characteristics and hence a different signal for a spectrophotometric reading (HogenEsch et al. 2018).

It is therefore not straightforward to predict the adsorption behaviour of a surrogate protein relative to the antigens contained in an adjuvanted vaccine. Protein adsorption can have an impact on aluminium hydroxide particle size, resulting in e.g. a different signal for a spectrophotometric reading. To determine aluminium concentration by spectrophotometry, the inventors believe the aluminium particles present in a calibration curve and in the sample should have substantially the same conformation to give rise to the same optical signal.

The inventors have discovered that certain proteins are able to act as surrogates for MenB antigens in standard compositions for the purposes of establishing the concentration of aluminium hydroxide concentration in MenB vaccines.

### SUMMARY OF THE INVENTION

As discussed above, the inventors have discovered that certain proteins are able to act as surrogates for MenB antigens in standard compositions for the purposes of establishing the concentration of aluminium hydroxide in MenB vaccines. Using these proteins in place of MenB antigens in standard compositions results in a highly efficient process for measuring aluminium hydroxide concentration.

Advantages of the embodiments of the invention relative to the prior art may be one of more of the following:
I. More efficient measurement of aluminium hydroxide concentration.
II. Faster measurement of aluminium hydroxide concentration.
III. Amenability to high throughput methods.
IV. Amenability to spectrophotometric plate reading.
V. Amenability to robotic liquid handling methods.
VI. Simplified handling processes by operators.
VII More accurate aluminium hydroxide concentration measurement.
VIII. Measurement of aluminium hydroxide concentration without interruption of continuous vaccine production.
IX. Reduce the number or quantity of reagents required.

In one aspect there is provided a standard composition comprising aluminium hydroxide and an antigen surrogate, in particular a MenB antigen surrogate, comprising ovalbumin, fosvitin, lactoferrin, or alpha-casein.

In another aspect there is provided a composition comprising aluminium hydroxide and ovalbumin. In a further aspect there is provided a composition comprising aluminium hydroxide and fosvitin. In another aspect there is provided a composition comprising aluminium hydroxide and lactoferrin. In another aspect there is provided a composition comprising aluminium hydroxide and alpha-casein.

In a further aspect there is provided the use of an antigen surrogate selected from ovalbumin, fosvitin, lactoferrin, or alpha-casein as a surrogate for one or more MenB antigens in a standard composition for measuring the concentration of aluminium hydroxide in a sample composition.

In another aspect there is provided the use of an antigen surrogate selected from ovalbumin, fosvitin, lactoferrin, or alpha-casein in a standard composition in a method of measuring the concentration of aluminium hydroxide in a sample composition wherein the sample composition comprises one or more MenB antigens.

In a further aspect there is provided a kit comprising a sample composition and one or more standard compositions, wherein the sample composition comprises one or more MenB antigen and the one or more standard compositions comprise the same components at the same concentrations as the sample composition except that the one or more standard compositions: (A) comprise (i) one or more known concentrations of aluminium hydroxide and (ii) a MenB antigen-surrogate selected from ovalbumin, fosvitin, lactoferrin, or alpha-casein and (B) do not comprise the one or more MenB antigen(s).

In another aspect there is provided a kit comprising one or more standard compositions, wherein the sample composition comprises one or more MenB antigens and the one or more standard compositions comprise the same components at the same concentrations as the sample composition except that the one or more standard compositions: (A) comprise (i) one or more known concentrations of aluminium hydroxide and (ii) a MenB antigen-surrogate selected from ovalbumin, fosvitin, lactoferrin, or alpha-casein and (B) do not comprise the MenB antigen.

In another aspect there is provided a method of measuring the concentration of aluminium hydroxide in a sample composition wherein the sample composition comprises one or more of MenB antigens, in particular one or more MenB antigens selected in the group consisting of polypeptide sharing at least about 90%, such as about 95%, such as about 99%, such as 100% identity with any one of SEQ ID NO: 1 to SEQ ID NO: 4 or SEQ ID NO: 9 to SEQ ID NO: 21, the method comprising:
(a) providing one or more standard compositions wherein the one or more standard compositions comprise the same components at the same concentrations as the sample composition except that (i) the one or more standard compositions, as defined herein, do not comprise the one or more MenB antigens present in the sample composition and instead comprise a MenB antigen surrogate comprising ovalbumin, fosvitin, lactoferrin, or alpha-casein and (ii) the one or more standard compositions comprise aluminium hydroxide at a known concentration,
(b) obtaining one or more signals from the one or more standard compositions, wherein the level of the signals correlate with the concentration of aluminium hydroxide present in the standard compositions,
(c) obtaining a signal from the sample composition in the same way as obtained for the one or more standard compositions in step (b) and
(d) comparing the level of the signal from the sample composition with the level of the one or more signals from the standard compositions to establish the concentration of aluminium hydroxide in the sample composition.

In another aspect there is provided a sample composition comprising aluminium hydroxide and one or more MenB antigens, wherein the concentration of aluminium hydroxide in the sample composition was measured using one or more standard compositions comprising an antigen surrogate selected from ovalbumin, fosvitin, lactoferrin, or alpha-casein as a surrogate for the one or more MenB antigens, wherein the one or more standard compositions are analogous to the sample composition.

In a further aspect there is provided a composition comprising aluminium hydroxide and one or more MenB antigens wherein the composition was produced in the same batch as the sample composition of the invention.

Further aspects of the invention will be evident from the detailed description below.

### BRIEF DESCRIPTION OF THE SEQUENCES

SEQ ID NO: 1 Polypeptide sequence of a MenB antigen 936-741
SEQ ID NO: 2 Polypeptide sequence of a MenB antigen 287-953
SEQ ID NO: 3 Polypeptide sequence of a MenB antigen 961c
SEQ ID NO: 4 Polypeptide sequence of PorA (Neisseria meningitidis strain NZ98/254)
SEQ ID NO: 5 Polypeptide sequence of chicken ovalbumin
SEQ ID NO: 6 Polypeptide sequence of bovine alpha-s1 casein
SEQ ID NO: 7 Polypeptide sequence of fosvitin
SEQ ID NO: 8 Polypeptide sequence of bovine lactotransferrin
SEQ ID NO: 9 Polypeptide sequence of a MenB antigen fHbp v1
SEQ ID NO: 10 Polypeptide sequence of the MenB antigen fHbp v1.13
SEQ ID NO: 11 Polypeptide sequence of the MenB antigen fHbp v1.13_E211A/S216R
SEQ ID NO: 12 Polypeptide sequence of the MenB antigen fHbp v1.15
SEQ ID NO: 13 Polypeptide sequence of the MenB antigen fHbp v1.55
SEQ ID NO: 14 Polypeptide sequence of a MenB antigen fHbp v2
SEQ ID NO: 15 Polypeptide sequence of a MenB antigen fHbp v2_S32V/L123R
SEQ ID NO: 16 Polypeptide sequence of a MenB antigen fHbp v3
SEQ ID NO: 17 Polypeptide sequence of a MenB antigen fHbp v3_S32V/L126R
SEQ ID NO: 18 Polypeptide sequence of the MenB antigen fHbp v3.45
SEQ ID NO: 19 Polypeptide sequence of a MenB antigen fHbp v2-v3-v1 #1
SEQ ID NO: 20 Polypeptide sequence of a MenB antigen fHbp v2-v3-v1 #2
SEQ ID NO: 21 Polypeptide sequence of a MenB antigen fHbp v2-v3-v1 #3 (fHbp 23S_1.13_E211A/E232A)

### BRIEF DESCRIPTION OF THE FIGURES

FIG. 1: Measurements of aluminium hydroxide concentration using established methodology.

FIG. 2: Spectra from MenB vaccine and blank, plus the ratio between spectra.

FIG. 3: Detected signal for ovalbumin compared to MenB vaccine.

FIG. 4: Detected signal for BSA compared to MenB vaccine.

FIG. 5: Detected signal for lysozyme compared to MenB vaccine.

FIG. 6: Detected signal for alpha-casein compared to MenB vaccine.

FIG. 7: Detected signal for dephosphorylated alpha-casein compared to MenB vaccine.

FIG. 8: Detected signal for fosvitin compared to MenB vaccine.

FIG. 9: Detected signal for alpha-lactalbumin compared to MenB vaccine.

FIG. 10: Detected signal for bovine haemoglobin compared to MenB vaccine.

FIG. 11: Detected signal for pepsin compared to MenB vaccine.

FIG. 12: Detected signal for lactoferrin compared to MenB vaccine.

FIG. 13: Detected signal for ovalbumin (with fitting), lactoferrin and fosvitin, (with fitting and subtraction) overlaid.

### DETAILED DESCRIPTION OF THE INVENTION

### Definitions

“Concentration of aluminium hydroxide” is used herein to refer to the content or amount of aluminium hydroxide, in particular, in a sample composition. As used herein, the term “sample composition” is intended to refer to a composition to be analysed, in which the concentration of aluminium hydroxide is unknown. In some embodiment, the sample composition is a solution, i.e., a composition in which the aluminium hydroxide is substantially dissolved. In some embodiment, the sample composition is a suspension, i.e., a composition in which the aluminium hydroxide is substantially in the form of undissolved particles.

“MenB antigen” is used herein to refer to a polypeptide that is capable of eliciting an immunological response against N. meningitidis serogroup B strain (MenB) and/or N. gonorrhoeae. As used herein, the term “polypeptide” may be understood as referring to either single protein or to a fusion of two or more proteins, e.g., the fusion of fHbp subtype proteins. As used herein, it is also understood that the MenB antigen comprises one or more epitope(s) against which the immune response is elicited.

“MenB vaccine” is used herein to refer to a composition, in particular an immunogenic composition, comprising one or more MenB antigens.

“Antigen surrogate” is used herein to refer to a polypeptide that mimics an antigen for the purpose of providing a standard composition. More specifically, the antigen surrogate is a MenB antigen surrogate. As used herein, the terms “antigen surrogate” and “surrogate antigen” can substitute one another. It is to be understood that where the term “antigen surrogate” is used in any paragraph of the instant description, it refers in particular aspects to “MenB antigen surrogate”. As used herein, “antigen surrogate” and “MenB antigen surrogate” can substitute one another.

The articles “a” and “an” are used herein to refer to one or to more than one (i.e., to at least one) of the grammatical object of the article. By way of example, “an element” means one element or more than one element.

“Or” supports, contemplates, and when recited in the claims, claims “one or a combination of” as in “one or a combination of A, B, or C.” To illustrate, “A, B, or C” means A alone, B alone, C alone, the combination of A and B, the combination of A and C, the combination of B and C, and the combination of A, B, and C, unless otherwise illustrated. That is, “or” supports and contemplates “and” as in “and/or” wherein “and/or” includes any combinations within the list of alternatives without being limited solely to the combination of all alternatives in a list (i.e. “A, B, or C” includes “A and B” and is not limited to “A, B, and C”).

Furthermore, the recitation of a list of alternatives, which may be conjoined by “and” and from which at least one alternative is selected, further contemplates and supports all combinations within the list of alternatives. For example, “X is selected from the group of: A, B, and C” contemplates and supports “X is selected from the group of: A, B, C, and combinations thereof,” “X is selected from at least one of the group of: A, B, and C,” and “X is selected from one or more of the group of: A, B, and C.” For further example, “X is selected from the group consisting of A, B, and C” contemplates and supports “X is selected from the group consisting of A, B, C, and combinations thereof,” “X is selected from at least one of the group consisting of A, B, and C,” or “X is selected from one or more of the group consisting of A, B, and C.”

Each of the following contemplates and supports any of the others: “comprises,” “consists of,” “consists essentially of,” “is/are/being,” “is selected from,” “is at least selected from,” “is selected from the group of,” “is selected from the group consisting of,” “is at least selected from the group consisting of,” “is from at least one of the group consisting of,” and “is from one or more of the group consisting of.” For example and in consideration of the above regarding combinations of listed elements, recitation of “X comprises an A, a B, or a C” in the specification contemplates and supports embodiments wherein “X consists of an A, a B, or a C,” “X consists of an A, a B, a C, or combinations thereof,” “X consists of one or more of an A, a B, or a C,” “X is one or more of an A, a B, or a C,” “X is an A, a B, a C, or combinations thereof,” “X is selected from an A, a B, or a C,” “X is selected from an A, a B, a C, or combinations thereof,” “X is selected from the group consisting of an A, a B, a C, and combinations thereof,” “X is selected from at least one of the group consisting of an A, a B, and a C,” or “X is selected from one or more of the group consisting of an A, a B, and a C.”

When a specific component of an embodiment is listed—e.g. “X comprises A, B, or C”—then also supported and contemplated are any embodiments which specifically exclude any individual or combinations of components—e.g. “X comprises A, but not B or C” or “X comprises A but does not comprise B or C.”

“About” as used herein when referring to a measurable value such as an amount, a temporal duration, a quantum of measurement, and the like, is meant to encompass variations of +/−20% or +/−10%, more preferably +/−5%, even more preferably +/−1%, and still more preferably +/−0.1% from the specified value, as such variations are appropriate to perform the disclosed methods.

### Standard Compositions and Surrogate Antigens

Many aspects of the invention involve one or more standard compositions. The one or more standard compositions substantially correspond to the sample composition but for (a) the substitution of the one or more MenB antigens in the sample composition for an antigen surrogate, in particular a MenB antigen surrogate, and (b) a known concentration of aluminium hydroxide being present in the standard composition, rather than the unknown concentration in the sample composition.

Without wishing to be bound by theory, it is believed that the antigen surrogate, in particular the MenB antigen surrogate, may adsorb to aluminium hydroxide and provide signals during analysis (such as spectrophotometry) in substantially the same manner as the substituted MenB antigens.

Ideally the one or more standard compositions are as similar as possible to the sample composition, but for the concentration of aluminium hydroxide (which is known for the one or more standard compositions, but unknown for the sample composition) and for the substitution of the one or more MenB antigens in the sample composition with the antigen surrogate, in particular the MenB antigen surrogate, in the one or more standard compositions.

In one embodiment, the antigen surrogate, in particular the MenB antigen surrogate, is ovalbumin. In an alternative embodiment, the antigen surrogate, in particular the MenB antigen surrogate, is alpha-casein. In a further embodiment, the antigen surrogate, in particular the MenB antigen surrogate, is fosvitin. In a yet further embodiment, the antigen surrogate, in particular the MenB antigen surrogate, is lactoferrin. Most suitably, the antigen surrogate, in particular the MenB antigen surrogate, is ovalbumin.

The ovalbumin may be ovalbumin from any organism which produces ovalbumin. For example, ovalbumin is the main protein found in egg white. Suitably, the ovalbumin is chicken ovalbumin (also called chicken egg albumin). Chicken ovalbumin is a protein of 385 amino acids in length, with a molecular weight of 42.7 kDa. The polypeptide sequence of chicken ovalbumin is Uniprot accession number P01012. In one embodiment, the ovalbumin shares at least about 70% identity, such as about 80% identity, such as about 90% identity, such as 100% identity, with this sequence (SEQ ID NO: 5). In one embodiment, the ovalbumin shares at least 70% identity, such as 80% identity, such as 90% identity, such as 100% identity, with this sequence (SEQ ID NO: 5). Within the scope of the present invention, the term “at least 70% identity” encompasses 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% identity.

In practice, the percentage of identity may be assessed according to any suitable and/or well-established protocols in the state of the art.

In one embodiment, the ovalbumin is full length ovalbumin, Accordingly, in one embodiment the ovalbumin is about 300 to about 450 amino acids in length, such as about 350 to about 420 amino acids in length, such as about 385 amino acids in length. In one embodiment, the ovalbumin is full length ovalbumin, Accordingly, in one embodiment the ovalbumin is 300 to 450 amino acids in length, such as 350 to 420 amino acids in length, such as about 385 amino acids in length. In a further embodiment, the ovalbumin is a fragment of full-length ovalbumin. In some embodiments, the fragment may be of at least about 100 amino acids in length, such as at least about 150, such as at least about 200, such as at least about 250, such as at least about 300 amino acids in length. The fragment may be up to about 420 amino acids in length, such as at up to about 390, such as up to about 360, such as up to about 330 amino acids in length. The fragment may be of at least 100 amino acids in length, such as at least 150, such as at least 200, such as at least 250, such as at least 300 amino acids in length. The fragment may be up to about 420 amino acids in length, such as at up to about 390, such as up to about 360, such as up to about 330 amino acids in length. The fragment may be up to 420 amino acids in length, such as at up to 390, such as up to 360, such as up to 330 amino acids in length.

In some embodiments, ovalbumin comprises N-terminal acetylation, potential phosphorylation at serine 68 and serine 344; and glycosylation at asparagine 292.

In some embodiments, ovalbumin belongs to one of three different subclasses: A1, containing two phosphate groups; A2, containing one phosphate group, and A3 containing no phosphate groups.

The alpha-casein may be alpha-casein from any organism which produces alpha-casein. For example, alpha-casein is a major constituent of milk. Suitably, the alpha-casein is bovine alpha-casein. Bovine alpha casein is divided into four subtypes: alpha-s1, alpha-s2, beta, and kappa. The polypeptide sequence of bovine alpha-s1 casein is Uniprot accession number P02662. In one embodiment, the alpha-s1 casein shares about 70%, such as about 80%, such as about 90%, such as 100% identity with this sequence (SEQ ID NO: 6). In one embodiment, the alpha-s1 casein shares 70%, such as 80%, such as 90%, such as 100% identity with this sequence (SEQ ID NO: 6).

In one embodiment the alpha-casein is full length alpha-casein. In one embodiment the alpha casein is about 200 to about 250 amino acids in length, such as about 210 to about 230 amino acids in length, such as about 214 amino acids in length, such as about 223 amino acids in length. Accordingly, in one embodiment the alpha casein is 200 to 250 amino acids in length, such as 210 to 230 amino acids in length, such as about 214 amino acids in length, such as about 223 amino acids in length. In a further embodiment, the alpha-casein is a fragment of full length alpha-casein. In some embodiments, the fragment may be of at least about 100 amino acids in length, such as at least about 150, such as at least about 200, such as at least about 210 amino acids in length. The fragment may be of at least 100 amino acids in length, such as at least about 150, such as at least 200, such as at least 210 amino acids in length.

Suitably the alpha-casein is bovine alpha-s1 casein. This type of alpha-casein exists as major and minor forms. The major form contains 8 phosphate groups, and the minor form contains 9 phosphate groups. Bovine alpha-s2 casein exists in four isoforms, having between 10 and 14 phosphate groups.

The fosvitin may be fosvitin from any organism which produces fosvitin. Fosvitin (also known as phosvitin) is a highly phosphorylated heterogenous protein which occurs naturally in egg yolk. Suitably the fosvitin is hen egg yolk fosvitin. This type of fosvitin is composed of 7 components, wherein the 2 main components (fosvitin-alpha and fosvitin-beta) account for 80% and 15% respectively. Suitably the fosvitin comprises about 50-100%, such as about 60-90%, such as about 80% fosvitin-alpha or comprises about 5-30%, such as about 10-20%, such as about 15% fosvitin-beta. Suitably the fosvitin comprises 50-100%, such as 60-90%, such as about 80% fosvitin-alpha or comprises 5-30%, such as 10-20%, such as about 15% fosvitin-beta. In one embodiment, the fosvitin comprises fosvitin-alpha. In another embodiment, the fosvitin comprises fosvitin-beta. The polypeptide sequence of fosvitin is Uniprot accession number P56530. In one embodiment, the fosvitin shares about 70%, such as about 80%, such as about 90%, such as 100% identity with this sequence (SEQ ID NO: 7). In one embodiment, the fosvitin shares 70%, such as 80%, such as 90%, such as 100% identity with this sequence (SEQ ID NO: 7).

In one embodiment the fosvitin is full length fosvitin. In one embodiment the fosvitin is about 200 to about 250 amino acids in length, such as about 210 to about 230 amino acids in length, such as about 217 amino acids in length. Accordingly, in one embodiment the fosvitin is 200 to 250 amino acids in length, such as 210 to 230 amino acids in length, such as about 217 amino acids in length. In a further embodiment, the fosvitin is a fragment of full length fosvitin. The fragment may be of at least about 100 amino acids in length, such as at least about 150, such as at least about 200 amino acids in length. The fragment may be of at least about 100 amino acids in length, such as at least 150, such as at least 200 amino acids in length.

Lactoferrin (also known as lactotransferrin (LTF)), is a multifunctional protein of the transferrin family. Suitably the lactoferrin is human, bovine or mouse lactoferrin. Most suitably the lactoferrin is bovine lactoferrin, such as bovine milk lactoferrin. Lactoferrin is a globular glycoprotein with a molecular mass of about 80 kDa that is widely represented in various secretory fluids, such as milk, saliva, tears, and nasal secretions. The polypeptide sequence of bovine lactoferrin is Uniprot accession number P24627. In one embodiment, the bovine lactoferrin shares about 70%, such as about 80%, such as about 90%, such as 100% identity with this sequence (SEQ ID NO: 8). In one embodiment, the bovine lactoferrin shares 70%, such as 80%, such as 90%, such as 100% identity with this sequence (SEQ ID NO: 8).

In one embodiment the lactoferrin is full length lactoferrin. In one embodiment the lactoferrin is about 600 to about 800 amino acids in length, such as about 650 to about 750 amino acids in length, such as about 700 amino acids in length. In one embodiment the lactoferrin is 600 to 800 amino acids in length, such as 650 to 750 amino acids in length, such as about 700 amino acids in length. In a further embodiment, the lactoferrin is a fragment of full length lactoferrin. The fragment may be of at least about 200 amino acids in length, such as at least about 300, such as at least about 400, such as at least about 500, such as at least about 600. The fragment may be of at least 200 amino acids in length, such as at least 300, such as at least 400, such as at least 500, such as at least 600.

The accuracy of concentration measurements may be improved by using a greater number of standard compositions. Suitably more than one standard composition is used and analysed to prepare signals, such that ideally a standard curve may be produced. Within the scope of the instant invention, the term “more than one” encompasses 2, 3, 4, 5, 6, 7, 8, 9, 10 and more.

In some embodiments, the antigen surrogate, in particular the MenB antigen surrogate, is phosphorylated, such as at least about 50% phosphorylated, such as completely phosphorylated. Suitably the antigen surrogate, in particular the MenB antigen surrogate, is phosphorylated, such as at least 50% phosphorylated, such as completely phosphorylated. Within the scope of the present invention, the term “at least 50% phosphorylated” encompasses 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% phosphorylated.

In one embodiment the one or more standard compositions further comprise sodium chloride, histidine, or sucrose. Suitably the one or more standard compositions consist essentially of an antigen surrogate, in particular a MenB antigen surrogate, aluminium hydroxide, sodium chloride, histidine, and sucrose, such as consisting of an antigen surrogate, in particular a MenB antigen surrogate, aluminium hydroxide, sodium chloride, histidine, and sucrose.

The surrogate antigen, in particular the MenB antigen surrogate, in the one or more standard compositions serves to replace Men B antigens. Accordingly, in one embodiment the one or more standard compositions do not comprise any MenB antigens. In some embodiments, the one or more standard compositions do not comprise any proteins other than the antigen surrogate, in particular the MenB antigen surrogate.

Suitably the concentration of the antigen surrogate, in particular the MenB antigen surrogate, is substantially, such as exactly, the same as the concentration of the one or more MenB antigens in the sample composition. Suitably the concentration is the same as the total concentration of all MenB antigens in the sample composition. In one embodiment, the concentration of the antigen surrogate, in particular the MenB antigen surrogate, is greater than about 50 mcg/ml, such as greater than about 200 mcg/ml. In one embodiment, the concentration of the antigen surrogate is about 200 to about 500 mcg/ml, such as about 250 mcg/ml. In one embodiment, the concentration of the antigen surrogate, in particular the MenB antigen surrogate, is greater than 50 mcg/ml, such as greater than 200 mcg/ml. In one embodiment, the concentration of the antigen surrogate is 200 to 500 mcg/ml, such as 250 mcg/ml. Within the scope of the present invention, the term “200 to 500 mcg/ml” encompasses about 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 and 500 mcg/ml.

Set concentrations of aluminium hydroxide are present in the one or more standard compositions. For optimal concentration measurement, a broad range of concentrations should be used across the standard compositions, with some below, some above, and some close to the suspected sample concentration. In some embodiments, the concentration of aluminium hydroxide in the standard compositions is about +/−100%, such as about +/−50%, around the suspected concentration of aluminium hydroxide in the sample composition. Suitably, the concentration of aluminium hydroxide in the standard compositions is +/−100%, such as +/−50%, around the suspected concentration of aluminium hydroxide in the sample composition.

In one embodiment, the one or more standard compositions comprise from about 0.1 to about 20 mg aluminium hydroxide per ml of standard composition, such as from about 0.5 to about 10 mg/ml, more suitably from about 0.7 to about 7 mg/ml, more especially from about 1.5 to about 4 mg/ml, and in particular around about 3 mg/ml. In further embodiments, three or more standard compositions are used and the concentration of aluminium hydroxide in at least one of the three or more standard compositions is around about 2.0 mg/ml, another of the three or more standard compositions is around about 3 mg/ml, and a third of the three or more standard compositions is around about 4 mg/ml. In yet further embodiments, 5 or more standard compositions are used and the standard compositions comprises around about 2.0 mg aluminium hydroxide per ml of standard composition, around about 2.5 mg/ml, around about 3 mg/ml, around about 3.5 mg/ml, and around about 4 mg/ml.

In one embodiment, the one or more standard compositions comprise from 0.1 to 20 mg aluminium hydroxide per ml of standard composition, such as from 0.5 to 10 mg/ml, more suitably from 0.7 to 7 mg/ml, more especially from 1.5 to 4 mg/ml, and in particular around 3 mg/ml. In further embodiments, three or more standard compositions are used and the concentration of aluminium hydroxide in at least one of the three or more standard compositions is around 2.0 mg/ml, another of the three or more standard compositions is around 3 mg/ml, and a third of the three or more standard compositions is around 4 mg/ml. In yet further embodiments, 5 or more standard compositions are used and the standard compositions comprises around 2.0 mg aluminium hydroxide per ml of standard composition, around 2.5 mg/ml, around 3 mg/ml, around 3.5 mg/ml, and around 4 mg/ml.

In some embodiments, the specification range for MenB vaccines of particular interest is about 2.4 mg/ml to about 3.6 mg/ml aluminium hydroxide. In some embodiments, the specification range for MenB vaccines of particular interest is 2.4 mg/ml to 3.6 mg/ml aluminium hydroxide. Within the scope of the present invention the term “2.4 mg/ml to 3.6 mg/ml” encompasses about 2.4, 2.5, 2.6, 2.7, 2.8, 2.9, 3.0, 3.1, 3.2, 3.3, 3.4, 3.5 and 3.6 mg/ml. The target concentration for MenB vaccines of particular interest is about 3.0 mg/ml, in particular 3.0 mg/ml. Obtaining batches of vaccines consistently containing concentrations of aluminium hydroxide within this range is desirable. To ensure this is achieved, the method of measuring aluminium hydroxide concentration must measure accurately around and within this concentration range.

The methods of the invention may be used with any volumes of standard compositions. In certain embodiments the volume of the one or more standard compositions is from about 0.1 to about 5.0 ml, such as from about 0.3 to about 3.0 ml, more suitably from about 0.1 to about 5.0 ml, or more suitably still from about 0.3 to about 3.0 ml. Nonetheless, in certain embodiments the volume of the one or more standard compositions is from 0.1 to 5.0 ml, such as from 0.3 to 3.0 ml, more suitably from 0.1 to 5.0 ml, or more suitably still from 0.3 to 3.0 ml. Within the scope of the present invention the term “from 0.1 to 5.0 ml” encompasses about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.2, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 and 5.0 ml.

In some embodiments the sample and/or the standard compositions further comprise one or more excipients, such as saline.

In one embodiment, the one or more standard compositions comprise, essentially consist of or consist of, the following components in the following concentration ranges, as set in Table 1:

TABLE 1

| Component | Concentration |
|---|---|
| Al(OH)₃ | 0.1 to 10 mg/ml |
| NaCl | 1 to 10 mg/ml |
| Histidine pH 6.3 | 5 to 15 mM |
| Surrogate antigen (Surrogate MenB antigen) | 50 to 500 mcg/ml |
| Sucrose | 0.5 to 5.0% w/v |

The methods of the invention may be used for substantially all concentrations of aluminium hydroxide expected/intended to be present in the sample composition. In some embodiments, the concentration of aluminium hydroxide in the sample composition is intended to be from about 0.1 to about 10.0 mg/ml, such as from about 1 to about 5 mg/ml, suitably from about 2 to about 4 mg/ml, more suitably around about 3 mg/ml. However, suitably the concentration of aluminium hydroxide in the sample composition is intended to be from 0.1 to 10.0 mg/ml, such as from 1 to 5 mg/ml, suitably from 2 to 4 mg/ml, more suitably around 3 mg/ml. Within the scope of the present invention, the term “0.1 to 10.0 mg/ml” encompasses 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

The term ‘standard composition’ is used herein merely as a label. The use of the word ‘standard’ is not intended to have any limiting effect on the composition.

### Sample Composition

Many aspects of the invention involve a sample composition. Methods of the invention may be used to measure the concentration of aluminium hydroxide in the sample composition.

In one embodiment the sample composition further comprises sodium chloride. In some embodiments, the sample composition comprises from about 1 to about 10 mg of sodium chloride per ml of sample composition (mg/ml), such as from about 4 to about 8 mg/ml, more suitably about about 6.25 mg/ml, most suitably about 6.25 mg/ml. Suitably the sample composition comprises from 1 to 10 mg of sodium chloride per ml of sample composition (mg/ml), such as from 4 to 8 mg/ml, more suitably about 6.25 mg/ml, most suitably 6.25 mg/ml.

In one embodiment the sample composition further comprises histidine. In some embodiments, the histidine is present at a concentration of about 1 to about 20 mM, such as about 5 to about 15 mM, more suitably around about 10 mM, most suitably about 10 mM. Suitably the histidine is present at a concentration of 1 to 20 mM, such as 5 to 15 mM, more suitably around 10 mM, most suitably 10 mM.

In a further embodiment, the sample composition further comprises sucrose. In some embodiments, the sucrose is present at a concentration of about 0.1 to about 10%, such as about 1 to about 3%, more suitably about 2.0% w of sucrose per volume of the sample composition (w/v). Suitably the sucrose is present at a concentration of 0.1 to 10%, such as 1 to 3%, more suitably about 2.0% w of sucrose per volume of the sample composition (w/v).

In some embodiments, the sample composition consists essentially of one or more MenB antigens. Suitably the sample composition consists essentially of one or more of MenB antigens 936-741, 287-953, 961c, and/or OMV. More suitably the sample composition consists of aluminium hydroxide, sodium chloride, histidine, sucrose, and one or more of MenB antigens 936-741, 287-953, 961c, and/or OMV.

Certain surrogate proteins, preferably surrogate antigens, in particular surrogate MenB antigens, may be selected depending on the expected concentration of aluminium hydroxide in the sample composition. For example, ovalbumin may be selected when concentrations of aluminium hydroxide are low, e.g. about 1.5-2 mg/ml. Alpha-casein may be selected when concentrations of aluminium hydroxide are high, e.g. about 3.5-4 mg/ml.

In one embodiment, the sample composition comprises, essentially consists of, or consists of, the following components in the following concentration ranges, as set in Tables 2 and 3:

TABLE 2

| Component | Concentration |
|---|---|
| Al(OH)₃ | 0.1 to 10 mg/ml |
| NaCl | 1 to 10 mg/ml |
| Histidine pH 6.3 | 5 to 15 mM |
| MenB antigen (per antigen) | 10 to 200 mcg/ml |
| Sucrose | 0.5 to 5.0% w/v |

TABLE 3

| Component | Concentration |
|---|---|
| Al(OH)₃ | 0.1 to 10 mg/ml |
| NaCl | 1 to 10 mg/ml |
| Histidine pH 6.3 | 5 to 15 mM |
| 936-741 | 50 to 200 mcg/ml |
| 961c | 50 to 200 mcg/ml |
| 287-953 | 50 to 200 mcg/ml |
| OMV | 10 to 100 mcg/ml |
| Sucrose | 0.5 to 5.0% w/v |

The term ‘sample composition’ is used herein merely as a label. The use of the word ‘sample’ is not intended to have any limiting effect on the composition.

### Aluminium Hydroxide

‘Aluminium hydroxide’ as used herein includes both hydroxides and oxyhydroxides (e.g. see chapters 8 & 9 of Vaccine Design (1995) eds. Powell & Newman. ISBN: 030644867X. Plenum). Mixtures of aluminium hydroxide and oxyhydroxides may be used, although it is preferred to use essentially Al(OH)₃. The aluminium hydroxide can take any suitable form (e.g. gel, crystalline, amorphous, etc.). Suitably the aluminium hydroxide is in powder form.

Suitably the aluminium hydroxide is selected from Al(OH)₃, AlO(OH), or a mixture thereof. More suitably the aluminium hydroxide consists essentially of, or more suitably consists of, Al(OH)₃.

Suitably the powder comprises Al(OH)₃ or AlO(OH). More suitably the powder consists essentially of, or more suitably consists of, Al(OH)₃.

It is to be understood that the aluminium hydroxide within the sample composition or the standard composition is either substantially dissolved or either in a particulate form (undissolved).

### MenB Antigens

In some embodiments, the sample composition comprises one or more MenB antigens. Within the scope of the present invention, the term “one or more” includes 1, 2, 3, 4, 5, 6, 7, 8, 9, 10 or more.

In some embodiments, the sample composition comprises two or more, in particular three or more, in particular four or more, more particularly five or more MenB antigens. In some embodiments, sample compositions comprising four or more MenB antigens are preferred.

In some embodiments, the MenB antigen is selected in the group consisting of a polypeptide sharing at least about 70% identity, such as at least about 80% identity, such as at least about 90% identity, such as at least about 95% identity, such as 100% identity, with any one of sequence SEQ ID NO: 1 to SEQ ID NO: 4 or SEQ ID NO: 9 to SEQ ID NO: 21. In some embodiments, the MenB antigen is selected in the group consisting of a polypeptide sharing at least 70% identity, such as at least 80% identity, such as at least 90% identity, such as at least 95% identity, such as 100% identity, with any one of sequence SEQ ID NO: 1 to SEQ ID NO: 4 or SEQ ID NO: 9 to SEQ ID NO: 21.

Non limitative examples of suitable Men B antigens are Men B antigens that have been extensively disclosed, e.g., in the following publications, which are herein incorporated by reference:
WO01/52885 (OUTER MEMBRANE VESICLE (OMV) VACCINE COMPRISING N. MENINGITIDIS SEROGROUP B OUTER MEMBRANE PROTEINS);
WO2003/010194 (MENINGOCOCCUS ADHESINS)
WO2003/020756 (HYBRID AND TANDEM EXPRESSION OF NEISSERIAL PROTEINS);
WO2004/048404 (MULTIPLE VARIANTS OF MENINGOCOCCAL PROTEIN NMB1870);
WO2007/060548 (CHIMERIC, HYBRID AND TANDEM POLYPEPTIDES OF MENINGOCOCCAL NMB1870);
WO2010/109323 (ADJUVANTING MENINGOCOCCAL FACTOR H BINDING PROTEIN);
WO2012/032489 (NON-LIPIDATED VARIANTS OF NEISSERIA MENINGITIDIS ORF2086 ANTIGENS);
WO2015/128480 (MODIFIED MENINGOCOCCAL FHBP POLYPEPTIDES);
WO2016/008960 (MODIFIED MENINGOCOCCAL FHBP POLYPEPTIDES);
WO2016/008961 (MENINGOCOCCUS VACCINES);
WO2018/042017 (VACCINES FOR NEISSERIA GONORRHOEAE);
WO2020/030782 (MODIFIED MENINGOCOCCAL FHBP POLYPEPTIDES);
WO2020/165711 (NEISSERIA MENINGITIDISCOMPOSITIONS AND METHODS THEREOF).

In some embodiments, the one or more MenB antigens is selected from the group comprising or consisting of fHbp, NadA, NHBA, OMV from N. meningitidis serogroup B, antigenic fragments thereof, functional mutants thereof, fusion proteins thereof and combinations thereof.

Within the scope of the present invention, the term “fusion protein” encompasses a fusion of the above antigens as well as a fusion with other accessory protein that enhances the immunogenicity of its fused counterpart.

In some embodiments, the sample composition comprises or consists of a combination of one or more fHbp antigens, a NadA antigen, a NHBA antigen and OMV from N. meningitidis serogroup B, antigenic fragments thereof, functional mutants thereof, and/or fusion proteins thereof.

### Hbp Antigen (Also Referred to as Protein 741)

In nature, the Factor H Binding Protein (741) is widely expressed on the N. meningitidis cell surface and binds to human factor H, a 150 kDa inhibitor of the alternative complement pathway (an innate component of the immune system's natural defence against infection that is not B- or T-cell mediated). The binding to factor H (fH) enhances the ability of N. meningitidis to resist complement-mediated killing, thereby providing an effective strategy to evade host defence mechanisms. Antibodies directed against fHbp can mediate serum bactericidal activity (e.g. direct bacteriolysis via the complement classical pathway) but also can block binding to fH. This blocking can increase the susceptibility to killing by the complement alternative pathway.

fHbp has been classified into three (main) variants 1 (v1), 2 (v2) and 3 (v3), which were further divided into sub-variants fHbp-1.x, fHbp-2.x and fHbp-3.x. Alternative fHbp classification schemes have been proposed (see the dedicated database with a unified fHbp nomenclature for the assignment of new sub-variants: (http)://neisseria.org/nm/typing/fhbp (also as (https)://pubmlst.org/neisseria/fHbp/)).

### MenB Antigen 936-741

In some embodiments, the MenB antigen is the MenB antigen 936-741.

MenB antigen 936-741 is a fusion protein product of proteins 936 and 741 (Factor H Binding Protein or fHbp). Protein 741 is the primary antigenic component while protein 936 is an accessory protein that enhances the immunogenicity of its fused counterpart.

Protein 936 is derived from N. meningitidis serogroup B Strain 2996 and Protein 741 from serogroup B Strain MC58. Proteins 936 and 741 are also identified in the literature as Genome-derived Antigens GNA 2091 (or NMB2091) and GNA 1870 (or NMB1870), respectively, based on genome mining or the reverse vaccinology approach. The fusion protein may be expressed via bacterial fermentation by standard recombinant DNA technology methods in E. coli using a plasmid vector system.

In one embodiment, the MenB antigen 936-741 comprises, or consist of, a polypeptide sharing at least about 70% identity, such as at least about 80% identity, such as at least about 90% identity, such as at least about 95% identity, such as 100% identity, with sequence SEQ ID NO: 1. In one embodiment, the MenB antigen 936-741 comprises, or consist of, a polypeptide sharing at least 70% identity, such as at least 80% identity, such as at least 90% identity, such as at least 95% identity, such as 100% identity, with sequence SEQ ID NO: 1.

In one embodiment, the MenB antigen 936-741 comprises, or consist of, the polypeptide sequence of SEQ ID NO: 1.

### Alternative Variants, Mutants and Fusions

In some embodiments, the MenB antigen comprises antigen fHbp v1, antigen fHbp v2 and/or antigen fHbp v3, mutated forms thereof and/or fusion forms thereof.

In some embodiments, antigen fHbp v1 comprises, or consist of, a polypeptide sharing at least about 70% identity, such as at least about 80% identity, such as at least about 90% identity, such as at least about 95% identity, such as 100% identity, with sequence SEQ ID NO: 9. In some embodiments, antigen fHbp v1 comprises, or consist of, a polypeptide sharing at least 70% identity, such as at least 80% identity, such as at least 90% identity, such as at least 95% identity, such as 100% identity, with sequence SEQ ID NO: 9.

In some embodiments, the Men B antigen comprises antigen fHbp v1.13, antigen fHbp v1.15 or a derivative thereof. In some embodiments, antigen fHbp v1.13 comprises, or consist of, a polypeptide sharing at least about 70% identity, such as at least about 80% identity, such as at least about 90% identity, such as at least about 95% identity, such as 100% identity, with sequence SEQ ID NO: 10. In some embodiments, antigen fHbp v1.13 comprises, or consist of, a polypeptide sharing at least 70% identity, such as at least 80% identity, such as at least 90% identity, such as at least 95% identity, such as 100% identity, with sequence SEQ ID NO: 10. In some embodiments, antigen fHbp v1.15 comprises, or consist of, a polypeptide sharing at least about 70% identity, such as at least about 80% identity, such as at least about 90% identity, such as at least about 95% identity, such as 100% identity, with sequence SEQ ID NO: 12. In some embodiments, antigen fHbp v1.15 comprises, or consist of, a polypeptide sharing at least 70% identity, such as at least 80% identity, such as at least 90% identity, such as at least 95% identity, such as 100% identity, with sequence SEQ ID NO: 12.

Derivatives of v1.13 (v1 with E211A, S216R, E232A mutations) and v1.15 are disclosed, e.g., in WO2020/030782 (MODIFIED MENINGOCOCCAL FHBP POLYPEPTIDES). Relevant sequences within the scope of the present invention can be found among sequences SEQ ID NO: 1-SEQ ID NO: 33 of WO2020/030782, which are incorporated herein by reference.

Illustratively, in some embodiments, antigen fHbp v1.13_E211A/S216R (variant v1.13 with mutations E211A and S216R) comprises, or consist of, a polypeptide sharing at least about 70% identity, such as at least about 80% identity, such as at least about 90% identity, such as at least about 95% identity, such as 100% identity, with sequence SEQ ID NO: 11. In some embodiments, antigen fHbp v1.13_E211A/S216R comprises, or consist of, a polypeptide sharing at least 70% identity, such as at least 80% identity, such as at least 90% identity, such as at least 95% identity, such as 100% identity, with sequence SEQ ID NO: 11.

Illustratively, in some embodiments, the MenB antigen comprises antigen fHbp v1.55. In some embodiments, antigen fHbp v1.55 comprises, or consist of, a polypeptide sharing at least about 70% identity, such as at least about 80% identity, such as at least about 90% identity, such as at least about 95% identity, such as 100% identity, with sequence SEQ ID NO: 13. In some embodiments, antigen fHbp v1.55 comprises, or consist of, a polypeptide sharing at least 70% identity, such as at least 80% identity, such as at least 90% identity, such as at least 95% identity, such as 100% identity, with sequence SEQ ID NO: 13.

In some embodiments, relevant sequences within the scope of the present invention may also be found among sequences SEQ ID NO: 1-SEQ ID NO: 74 of WO2020/165711 (NEISSERIA MENINGITIDISCOMPOSITIONS AND METHODS THEREOF), which are incorporated herein by reference.

In some embodiments, antigen fHbp v2 comprises, or consist of, a polypeptide sharing at least about 70% identity, such as at least about 80% identity, such as at least about 90% identity, such as at least about 95% identity, such as 100% identity, with sequence SEQ ID NO: 14. In some embodiments, antigen fHbp v2 comprises, or consist of, a polypeptide sharing at least 70% identity, such as at least 80% identity, such as at least 90% identity, such as at least 95% identity, such as 100% identity, with sequence SEQ ID NO: 14.

Mutations of antigen fHbp v2 may include a mutation in any one of amino acid residues S32, V33, L39, L41, F69, V100, 1113, F122, L123, V124, S125, G126, L127, G128, S151, H239, and E240.

Illustratively, in some embodiments, the MenB antigen comprises antigen fHbp-v2_S32V/L123R (variant v2 with mutations S32V and L123R). In some embodiments, antigen fHbp fHbp-v2_S32V/L123R comprises, or consist of, a polypeptide sharing at least about 70% identity, such as at least about 80% identity, such as at least about 90% identity, such as at least about 95% identity, such as 100% identity, with sequence SEQ ID NO: 15. In some embodiments, antigen fHbp fHbp-v2_S32V/L123R comprises, or consist of, a polypeptide sharing at least 70% identity, such as at least 80% identity, such as at least 90% identity, such as at least 95% identity, such as 100% identity, with sequence SEQ ID NO: 15.

In some embodiments, antigen fHbp v3 comprises, or consist of, a polypeptide sharing at least about 70% identity, such as at least about 80% identity, such as at least about 90% identity, such as at least about 95% identity, such as 100% identity, with sequence SEQ ID NO: 16. In some embodiments, antigen fHbp v3 comprises, or consist of, a polypeptide sharing at least 70% identity, such as at least 80% identity, such as at least 90% identity, such as at least 95% identity, such as 100% identity, with sequence SEQ ID NO: 16.

In some embodiments, the MenB antigen comprises antigen fHbp-v3.45. In some embodiments, antigen fHbp v3.45 comprises, or consist of, a polypeptide sharing at least about 70% identity, such as at least about 80% identity, such as at least about 90% identity, such as at least about 95% identity, such as 100% identity, with sequence SEQ ID NO: 18. In some embodiments, antigen fHbp v3.45 comprises, or consist of, a polypeptide sharing at least 70% identity, such as at least 80% identity, such as at least 90% identity, such as at least 95% identity, such as 100% identity, with sequence SEQ ID NO: 18. In some embodiments, relevant sequences within the scope of the present invention may also be found among sequences SEQ ID NO: 1-SEQ ID NO: 74 of WO2020/165711 (NEISSERIA MENINGITIDISCOMPOSITIONS AND METHODS THEREOF), which are incorporated herein by reference.

Mutations of antigen fHbp v3 may include a mutation in any one of amino acid residues S32, V33, L39, L41, F72, V103, T116, F125, L126, V127, S128, G129, L130, G131, S154, H242, and/or E243 for antigen fHbp v3.

Illustratively, in some embodiments, the MenB antigen comprises antigen fHbp-v3_S32V/L126R (variant v3 with mutations S32V and L126R). In some embodiments, antigen fHbp v3.45_S32V/L126R comprises, or consist of, a polypeptide sharing at least about 70% identity, such as at least about 80% identity, such as at least about 90% identity, such as at least about 95% identity, such as 100% identity, with sequence SEQ ID NO: 17. In some embodiments, antigen fHbp v3.45_S32V/L126R comprises, or consist of, a polypeptide sharing at least 70% identity, such as at least 80% identity, such as at least 90% identity, such as at least 95% identity, such as 100% identity, with sequence SEQ ID NO: 17.

Relevant sequences within the scope of the present invention, including the variants of antigens fHbp v2 and fHbp v3 can be found among sequences SEQ ID NO: 1-SEQ ID NO: 61 of WO2015/128480 (MODIFIED MENINGOCOCCAL FHBP POLYPEPTIDES) and SEQ ID NO: 1-SEQ ID NO: 60 of WO2016/008960 (MODIFIED MENINGOCOCCAL FHBP POLYPEPTIDES), which are incorporated herein by reference.

In some embodiments, the MenB antigen comprises a combination of antigen fHbp v1.55 and antigen fHbp-v3.45.

In some embodiments, antigen fHbp v1, antigen fHbp v2 and antigen fHbp v3 are fused together and are comprised in one single polypeptide. Examples of such fusion protein includes the fHbp v2-v3-v1 fusion protein. In some embodiments, fHbp v2-v3-v1 comprises, or consist of, a polypeptide sharing at least about 70% identity, such as at least about 80% identity, such as at least about 90% identity, such as at least about 95% identity, such as 100% identity, with sequence SEQ ID NO: 19, SEQ ID NO: 20 or SEQ ID NO: 21. In some embodiments, fHbp v2-v3-v1 comprises, or consist of, a polypeptide sharing at least 70% identity, such as at least 80% identity, such as at least 90% identity, such as at least 95% identity, such as 100% identity, with sequence SEQ ID NO: 19, SEQ ID NO: 20 or SEQ ID NO: 21.

Illustratively, SEQ ID NO: 19 and SEQ ID NO: 20 are disclosed, e.g., as SEQ ID NO: 48 and SEQ ID NO: 47, respectively, of WO2015/128480 (see above). Additional fHbp v2-v3-v1 fusion protein are also disclosed as sequences SEQ ID NO: 27-SEQ ID NO: 30 of WO2016/008960 (see above).

In some embodiments, the fHbp antigen is not lipidated. In some embodiments, the fHbp antigen is lipidated.

### NHBA Antigen (Also Referred to as Protein 287)

In some embodiment, the MenB antigen comprises NHBA antigen, a mutated form thereof and/or a fusion form thereof.

In nature, NHBA is expressed on the N. meningitidis cell surface. NHBA is able to bind heparin, and heparan sulphate. The binding to heparin is associated with an increased resistance to the killing activity of normal human sera. NHBA is derived from N. meningitidis serogroup B Strain NZ98/254 (also referred to as 394/98).

In some embodiments, the MenB antigen is the MenB antigen 287-953.

MenB antigen 287-953 is a fusion protein product of protein 287 (Neisserial Heparin Binding Antigen or NHBA) and protein 953. Protein 287 is the primary antigenic component while protein 953 is an accessory protein that enhances the immunogenicity of its fused counterpart.

Protein 953 is derived from serogroup B Strain 2996. NHBA and 953 are also identified in the literature as Genome-derived Antigens GNA2132 (or NMB2132) and GNA 1030 (or NMB1030), respectively, based on genome mining or the reverse vaccinology approach. The fusion protein may be expressed via bacterial fermentation by standard recombinant DNA technology methods in E. coli using a plasmid vector system.

In one embodiment, the MenB antigen 287-953 comprises, or consist of, a polypeptide sharing at least about 70% identity, such as at least about 80% identity, such as at least about 90% identity, such as at least about 95% identity, such as 100% identity, with sequence SEQ ID NO: 2. In one embodiment, the MenB antigen 287-953 comprises, or consist of, a polypeptide sharing at least 70% identity, such as at least 80% identity, such as at least 90% identity, such as at least 95% identity, such as 100% identity, with sequence SEQ ID NO: 2.

In one embodiment, the MenB antigen 287-953 comprises, or more suitably consist of, the polypeptide sequence of SEQ ID NO: 2.

### NadA Antigen (Also Referred to as Protein 961)

In some embodiment, the MenB antigen comprises NadA antigen, a mutated form thereof and/or a fusion form thereof.

In some embodiments, the MenB antigen is MenB antigen 961c.

MenB antigen 961c is a fragment of NadA (Neisseria adhesin A) protein, a surface-exposed oligomeric protein belonging to the Oligomeric Coiled-coil Adhesin (OCA) family involved in binding to epithelial cells.

MenB antigen 961c is a surface-exposed oligomeric protein belonging to the Oligomeric Coiled-coil Adhesin (OCA) family. It is a meningococcal adhesin molecule involved in binding to epithelial cells. The protein derives from N. meningitidis serogroup B Strain 2996. This protein is also known as Genome derived Antigen (GNA) 1994 (or NMB1994), based on the reverse vaccinology approach that allowed its identification. The MenB 961c antigen may be expressed via bacterial fermentation by standard recombinant DNA technology methods in E. coli using a plasmid vector system.

In one embodiment, the MenB antigen 961c comprises, or consist of, a polypeptide sharing at least about 70% identity, such as at least about 80% identity, such as at least about 90% identity, such as at least about 95% identity, such as 100% identity, with sequence SEQ ID NO: 3. In one embodiment, the MenB antigen 961c comprises, or consist of, a polypeptide sharing at least 70% identity, such as at least 80% identity, such as at least 90% identity, such as at least 95% identity, such as 100% identity, with sequence SEQ ID NO: 3.

In one embodiment, MenB antigen 961c comprises, or more suitably consist of, the polypeptide sequence of SEQ ID NO: 3.

### MenB Antigen OMV

In some embodiments, the MenB antigen is MenB antigen OMV

MenB antigen OMV are Outer Membrane Vesicles (OMV) from Neisseria meningitidis group B, most suitably strain NZ98/254. The primary immunogenic components of the OMV are the outer membrane proteins (OMPs) and the membrane-bound lipopolysaccharides (LPS). As OMVs are complex mixtures of lipids, OMPs, and periplasmic components, structural descriptions are limited to those that are likely related to the mode of action, namely the major OMP antigens and LPS. The major outer membrane proteins (OMPs) of N. meningitidis have been designated Class 1 (PorA, Serotype P1.4) through Class 5 (Opa). The Class 1, 2, and 3 proteins are porins that show significant antigenic variation. All meningococcal strains carry either Class 2 (PorB2) or Class 3 (Por B3) protein. These proteins function as anion-selective porins and probably occur in the outer membrane as trimers. A Class 4 OMP, also called Rmp (Reduction-modifiable protein) due to its shift in mobility in SDS-PAGE after reduction, is closely related to protein III (PIII) of N. gonorrhoeae. The Class 4/RmpM OMP is constitutively expressed, is antigenically invariable, and is closely associated with the porin molecules, acting as a stabilizer. The Class 5 protein, Opc, is a surface-exposed protein forming trimers or tetramers in the outer membrane. The N. meningitidis outer membrane is an asymmetric bi-layer that consists of phospholipids on the inner leaflet and the lipid anchor region of lipopolysaccharides (LPS), lipid A, on the outer leaflet. Meningococcal LPS is structurally distinct from those of enteric Gram-negative bacilli. It lacks the O antigen repeat present in enteric bacteria, but the core LPS molecule is heterogeneous both within and between strains and has been classified into 12 distinct immunotypes (L1-L12) on the basis of monoclonal antibody reactivity. OMV NZ vaccine contains both the L1 and L3 immunotypes. One-dimensional SDS-PAGE of OMV vaccines generally reveals between 20-30 proteins, with estimated molecular weights from 22,800 to 89, 100 Da. Among these are the highly expressed porin proteins (PorA and PorB), the Class 4 and 5 antigens and other major antigens, including Omp85, FetA (Class 1, previously referred to as FrpB), and NspA (Class 3).

In one embodiment, OMV may be measured as amount of total protein containing PorA P1.4. PorA is known to be the most immunogenic protein targeted by antibodies following vaccination with group B meningococcal OMV vaccines (Tappero et al 1999, van der Voort et al. 1996). A combination of biochemical and immunological approaches have demonstrated that the PorA protein has a beta-barrel structure and that most of the peptide sequence variation is located in two variable regions (VR1 and VR2), which correspond to surface-exposed loops I and IV of the proposed protein structure. The VR1 of the New Zealand strain from which the vaccine OMV is prepared is designated P1.7-2, and the VR2 is designated P1.4. The sequence of PorA (Neisseria meningitidis strain NZ98/254) is shown below (SEQ ID NO: 4).

In one embodiment, OMV comprises, or consist of, a polypeptide sharing at least about 70% identity, such as at least about 80% identity, such as at least about 90% identity, such as at least about 95% identity, such as 100% identity, with sequence SEQ ID NO: 4. In one embodiment, OMV comprises, or consist of, a polypeptide sharing at least 70% identity, such as at least 80% identity, such as at least 90% identity, such as at least 95% identity, such as 100% identity, with sequence SEQ ID NO: 4.

In one embodiment, OMV comprises, or more suitably consist of, the polypeptide sequence of SEQ ID NO: 4.

In one embodiment MenB antigen 936-741 (SEQ ID NO: 1) is present in the sample composition. In one embodiment MenB antigen 287-953 (SEQ ID NO: 2) is present in the sample composition. In one embodiment MenB antigen 961c (SEQ ID NO: 3) is present in the sample composition. In one embodiment MenB antigen OMV (SEQ ID NO: 4) is present in the sample composition. In some embodiments, the sample composition comprises 936-741 MenB antigen (SEQ ID NO: 1), 287-953 MenB antigen (SEQ ID NO: 2), 961c MenB antigen (SEQ ID NO: 3), or OMV MenB antigen (SEQ ID NO: 4). In one embodiment MenB antigens 936-741 (SEQ ID NO: 1), 287-953 (SEQ ID NO: 2), 961c (SEQ ID NO: 3) and/or OMV (SEQ ID NO:) are present in the sample composition. Suitably all of MenB antigens 936-741 (SEQ ID NO: 1), 287-953 (SEQ ID NO: 2), 961c (SEQ ID NO: 3) and OMV (SEQ ID NO: 4) are present in the sample composition.

In one embodiment each MenB antigen present in the sample composition is present at a concentration of about 10 to about 200 mcg/ml, such as about 5 to about 150 mcg/ml, more suitably as about 25 to about 50 mcg/ml. In one embodiment each MenB antigen present in the sample composition is present at a concentration of 10 to 200 mcg/ml, such as 5 to 150 mcg/ml, more suitably as 25 to 50 mcg/ml.

In one embodiment each MenB antigen present in the sample composition is present at a concentration of about 50 to about 150 mcg/ml, such as about 75 to about 125 mcg/ml, more suitably about 100 mcg/ml. In one embodiment each MenB antigen present in the sample composition is present at a concentration of 50 to 150 mcg/ml, such as 75 to 125 mcg/ml, more suitably about 100 mcg/ml. In one embodiment some MenB antigens present in the sample composition are present at a concentration of about 10 to about 100 mcg/ml, such as about 5 to about 50 mcg/ml, more suitably about 25 mcg/ml. In one embodiment some MenB antigens present in the sample composition are present at a concentration of 10 to 100 mcg/ml, such as 5 to 50 mcg/ml, more suitably 25 mcg/ml.

Suitably MenB antigens 936-741, 287-953, 961c or OMV each comprise or consist of a full length 936-741, 287-953, 961c, or OMV MenB polypeptide, respectively, or an immunogenic fragment of variable thereof.

Suitably MenB antigens 936-741, 287-953, 961c, or OMV each comprise or consist of a polypeptide sharing at least 90%, such as 95%, such as 99%, such as 100% identity with SEQ ID NO: 1, SEQ ID NO: 2, SEQ ID NO: 3 or SEQ ID NO: 4 respectively.

Suitably MenB antigens comprise or consist of a polypeptide sharing at least about 90%, such as about 95%, such as about 99%, such as 100% identity with SEQ ID NO: 1, SEQ ID NO: 2, SEQ ID NO: 3, SEQ ID NO: 4, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21. Suitably MenB antigens comprise or consist of a polypeptide sharing at least 90%, such as 95%, such as 99%, such as 100% identity with SEQ ID NO: 1, SEQ ID NO: 2, SEQ ID NO: 3, SEQ ID NO: 4, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21.

Suitably the one or more MenB antigens are produced by recombinant DNA technology, such as by production in E. coli cells.

In some embodiments, the one or more MenB antigens are suitably substantially adsorbed to the aluminum adjuvant in the sample composition, in particular the aluminium hydroxide adjuvant.

In some embodiments, the one or more MenB antigens are suitably substantially adsorbed to the aluminium hydroxide in the sample composition, such as completely adsorbed.

In some embodiments, the one or more MenB antigens in the sample composition are present in a concentration range within +/−30% of the desired concentration.

### Other Components

### Additional Antigens

In some embodiments, the sample composition may further include one or more additional antigens from N. meningitidis. In some embodiments, the one or more additional antigens include, without being limited to, capsular polysaccharide antigens, in particular conjugated capsular polysaccharide antigens. Non limited examples of conjugated capsular saccharide antigens include capsular saccharide from N. meningitidis serogroup A, capsular saccharide from N. meningitidis serogroup C, capsular saccharide from N. meningitidis serogroup W135 and capsular saccharide from N. meningitidis serogroup Y. Capsular saccharide from N. meningitidis serogroups A, C, W135 and Y are disclosed, e.g., in WO03/007985 (CAPSULAR POLYSACCHARIDE SOLUBILISATION AND COMBINATION VACCINES).

In some embodiments, in addition to the one or more MenB antigen, the sample composition further comprises capsular saccharide from N. meningitidis serogroup A (also referred herein as MenA antigen), capsular saccharide from N. meningitidis serogroup C (also referred herein as MenC antigen), capsular saccharide from N. meningitidis serogroup W135 (also referred herein as MenW135 antigen) and/or capsular saccharide from N. meningitidis serogroup Y (also referred herein as MenY antigen). In some embodiments, in addition to the one or more MenB antigen, the sample composition further comprises capsular saccharide from N. meningitidis serogroup A, capsular saccharide from N. meningitidis serogroup C, capsular saccharide from N. meningitidis serogroup W135 and capsular saccharide from N. meningitidis serogroup Y.

In some embodiments, capsular saccharide from N. meningitidis serogroup A may be modified so as to improve its stability. Example of such modifications may be found, e.g., in WO2020/182635 (CARBOCYCLIC DERIVATIVES AND CONJUGATED DERIVATIVES THEREOF, AND THEIR USE IN VACCINES). In some embodiments, in addition to the one or more MenB antigen, the sample composition further comprises a modified capsular saccharide from N. meningitidis serogroup A, capsular saccharide from N. meningitidis serogroup C, capsular saccharide from N. meningitidis serogroup W135 and capsular saccharide from N. meningitidis serogroup Y.

In some embodiments, the capsular saccharides may be conjugated to a carrier protein, including, but not limited to, bacterial toxins or toxoids, such as diphtheria or tetanus toxoids. In some embodiments, the carrier is CRM197 diphtheria toxoid. In some embodiments, in addition to the at least one MenB antigen, the sample composition further comprises capsular saccharide from N. meningitidis serogroup A conjugated to CRM197, capsular saccharide from N. meningitidis serogroup C conjugated to CRM197, capsular saccharide from N. meningitidis serogroup W135 conjugated to CRM197 and/or capsular saccharide from N. meningitidis serogroup Y conjugated to CRM197. In some embodiments, in addition to the at least one MenB antigen, the sample composition further comprises capsular saccharide from N. meningitidis serogroup A conjugated to CRM197, capsular saccharide from N. meningitidis serogroup C conjugated to CRM197, capsular saccharide from N. meningitidis serogroup W135 conjugated to CRM197, and capsular saccharide from N. meningitidis serogroup Y conjugated to CRM197.

Non limitative examples of such conjugated capsular saccharides suitable for the invention may be found in meningitidis commercial products such as, e.g., MENVEO, MENACTRA and NIMENRIX.

In some embodiments, the sample composition may further include one or more additional antigens from Streptococcus pneumoniae. Non limitative examples of such antigens may be found in commercial products such as, e.g, PREVNAR and SYNFLORIX.

In some embodiments, the sample composition may further include one or more additional antigens hepatitis B virus, such as, e.g., the surface antigen HBsAg.

In some embodiments, the sample composition may further include one or more additional antigens from Bordetella pertussis, such as, e.g., pertussis holotoxin (PT) and filamentous haemagglutinin (FHA), optionally in combination with pertactin and/or agglutinogens 2 and 3.

In some embodiments, the sample composition may further include one or more diphtheria antigens, such as, e.g., a diphtheria toxoid.

In some embodiments, the sample composition may further include one or more tetanus antigens, such as, e.g., a tetanus toxoid.

In some embodiments, the sample composition may further include one or more saccharide antigens from Haemophilus influenzae B (Hib), typically conjugated.

In some embodiments, the sample composition may further include one or more inactivated poliovirus antigens.

In some embodiments, the sample composition comprises, essentially consists of, or consists of a combination of MenB antigens 936-741, 287-953, 961c, OMV, capsular saccharide from N. meningitidis serogroup A, capsular saccharide from N. meningitidis serogroup C, capsular saccharide from N. meningitidis serogroup W135 and capsular saccharide from N. meningitidis serogroup Y.

In some embodiments, the sample composition comprises, essentially consists of, or consists of a combination of MenB antigens 936-741 (SEQ ID NO: 1), 287-953 (SEQ ID NO: 2), 961c (SEQ ID NO: 3), OMV (SEQ ID NO: 4), (ii) capsular saccharide from N. meningitidis serogroup A conjugated to CRM197, capsular saccharide from N. meningitidis serogroup C conjugated to CRM197, capsular saccharide from N. meningitidis serogroup W135 conjugated to CRM197, and capsular saccharide from N. meningitidis serogroup Y conjugated to CRM197.

In some embodiments, the sample composition comprises, essentially consists of, or consists of a combination of MenB antigens 936-741 (SEQ ID NO: 1), 287-953 (SEQ ID NO: 2), 961c (SEQ ID NO: 3), OMV (SEQ ID NO: 4), a fHbp v2-v3-v1 fusion protein, capsular saccharide from N. meningitidis serogroup A conjugated to CRM197, capsular saccharide from N. meningitidis serogroup C conjugated to CRM197, capsular saccharide from N. meningitidis serogroup W135 conjugated to CRM197, and capsular saccharide from N. meningitidis serogroup Y conjugated to CRM197.

Suitably the compositions will have a pharmaceutically acceptable osmolarity. A pharmaceutically acceptable osmolality will generally mean that solutions will have an osmolality which is approximately isotonic or mildly hypertonic.

In some embodiments, the compositions of the invention have a pH of about 4 to about 7, such as about 5 to about 6.5, more suitably about 5.5 to about 6. Suitably the compositions of the invention have a pH of 4 to 7, such as 5 to 6.5, more suitably 5.5 to 6. Within the scope of the present invention, the term “pH of 4 to 7” encompasses about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 and 7.0.

### Concentration Measurement

Greater accuracy in aluminium hydroxide concentration measurement may be achieved by using a greater number of standard compositions. In one embodiment, a standard curve is produced from analysis of the standard compositions. This way, the level of the signal from the sample composition is compared with the standard curve to establish the concentration of aluminium hydroxide in the sample composition.

Furthermore, greater accuracy in aluminium hydroxide concentration measurement may be achieved by using one or more standard compositions comprising a concentration of antigen surrogate which is close, or identical to, the concentration of the one or more MenB antigens in the sample composition. In one embodiment the concentration of the antigen surrogate, in particular the MenB antigen surrogate, in the one or more standard compositions is within about +/−10%, such as about +/−5%, more suitably within about +/−1%, especially within about +/−0.1% of the concentration of the one or more MenB antigens in the sample composition. In one embodiment the concentration of the antigen surrogate, in particular the MenB antigen surrogate, in the one or more standard compositions is within +/−10%, such as +/−5%, more suitably within +/−1%, especially within +/−0.1% of the concentration of the one or more MenB antigens in the sample composition.

In some embodiments, the aluminium hydroxide in the one or more standard compositions is in at least one of the mass to volume amounts of Table 13 or Table 14.

To perform a concentration measurement, one or more signals must be obtained from the sample composition and the one or more standard compositions. The signals from the sample composition are compared to those of the one or more standard compositions to establish the concentration of aluminium hydroxide in the sample composition.

In one embodiment at least 2 signals are obtained from each of at least 2 standard compositions, wherein each of the standard compositions has a different concentration of aluminium hydroxide. More suitably, at least 3 signals are obtained from each of at least 3 standard compositions, wherein each of the standard compositions has a different concentration of aluminium hydroxide. More suitably, at least 4 signals are obtained from each of at least 4 standard compositions, wherein each of the standard compositions has a different concentration of aluminium hydroxide.

More suitably, at least 5 signals are obtained from each of at least 5 standard compositions, wherein each of the standard compositions has a different concentration of aluminium hydroxide.

The signals may be obtained using any suitable analytical method which provides a signal from the sample composition and the one or more standard compositions. The level of the signal must correlate with the concentration of aluminium hydroxide present in the compositions. Suitable signals include those captured using electromagnetic spectroscopy, such as spectrophotometry. Accordingly, in one embodiment the signals are established using electromagnetic spectroscopy, more suitably spectrophotometry. The signals may be absorbance, transmittance or reflectance, most suitably absorbance.

In one embodiment, the method of the invention includes step (b) (ii) after step (b) and before step (c), wherein regression (e.g. estimation of the best straight line) is performed on the signals from the standard compositions, and wherein in step (d) the concentration of aluminium hydroxide in the sample composition is established from the regression.

It has been found that measurement accuracy can be improved if the electromagnetic radiation used when performing absorbance spectrophotometry suitably has a wavelength of about 400 nm, in particular 400 nm. Accordingly, in one embodiment the electromagnetic radiation used when performing absorbance spectrophotometry has a wavelength of about 1 to about 3,000 nm, more suitably about 300 to about 1,000 nm, such as about 350 to about 800 nm, in particular about 380 to about 600 nm, more particularly about 390 to about 500 nm, more especially about 395 to about 450 nm, most suitably about 400 nm. Accordingly, in one embodiment the electromagnetic radiation used when performing absorbance spectrophotometry has a wavelength of 1 to 3,000 nm, more suitably 300 to 1,000 nm, such as 350 to 800 nm, in particular 380 to 600 nm, more particularly 390 to 500 nm, more especially 395 to 450 nm, most suitably about 400 nm.

In one embodiment the spectrophotometry is performed at about 10 to about 30° C. In one embodiment the spectrophotometry is performed at 10 to 30° C., such as about 20° C. Within the scope of the present invention, the term “10 to 30° C.” encompasses about 10, 11, 12, 13, 14, 15, 16, 17, 18, 19, 20, 21, 22, 23, 24, 25, 26, 27, 28, 29 and 30° C.

Spectrophotometry is ideally performed during, or shortly after, the sample composition and the one or more standard compositions have been agitated sufficiently to achieve a homogenous mixture of components in the containers containing the compositions. In some embodiments, the electromagnetic spectroscopy is performed shortly, such as immediately, after shaking the compositions for about 1 to about 5 minutes at about 500 to about 2,000 rpm. Suitably the electromagnetic spectroscopy is performed shortly, such as immediately, after shaking the compositions for 1 to 5 minutes at 500 to 2,000 rpm.

Since aluminium hydroxide is an opalescent suspension and because of the absence of a single clear absorption peak, the ratio between the aluminium suspension spectra and its blank allows the identification of the wavelength at which the optical signal of the selected sample shows the higher significative response compared to its blank within the visible range. Accordingly, in one embodiment the method of the invention comprises the step of establishing the ratio between the aluminium suspension spectra and its blank and identifying the wavelength at which the optical signal of the selected sample shows the higher significative response compared to its blank within the visible range.

For optimal concentration measurement accuracy, signals should be obtained in the same manner, using the same parameters, under the same conditions, for the sample composition and the one or more standard compositions.

In one embodiment the method consists essentially of, or more suitably consists of, the recited steps.

### CLAUSES

Clauses setting out further embodiments of the invention are as follows:
1. A standard composition comprising an antigen surrogate, in particular a MenB antigen surrogate, selected from ovalbumin, fosvitin, lactoferrin or alpha-casein, and aluminium hydroxide.
2. Use of an antigen surrogate selected from ovalbumin, fosvitin, lactoferrin or alpha-casein as a surrogate for one or more MenB antigens in a standard composition for measuring the concentration of aluminium hydroxide in a sample composition.
3. Use of an antigen surrogate, in particular a MenB antigen surrogate, selected from ovalbumin, fosvitin, lactoferrin or alpha-casein in a standard composition in a method of measuring the concentration of aluminium hydroxide in a sample composition wherein the sample composition comprises one or more MenB antigens.
4. A kit comprising a sample composition and one or more standard compositions, wherein the sample composition comprises one or more MenB antigens and the one or more standard compositions comprise the same components at the same concentrations as the sample composition except that the one or more standard compositions comprise (i) one or more known concentrations of aluminium hydroxide and (ii) an antigen surrogate, in particular a MenB antigen surrogate, selected from ovalbumin, fosvitin, lactoferrin or alpha-casein, and wherein the standard composition does not comprise MenB antigens.
5. A method of measuring the concentration of aluminium hydroxide in a sample composition wherein the sample composition comprises one or more of MenB antigens, in particular one or more MenB antigens selected in the group consisting of polypeptide sharing at least 90%, such as 95%, such as 99%, such as 100% identity with SEQ ID NO: 1 to SEQ ID NO: 4, and SEQ ID NO: 9 to SEQ ID NO: 21, in particular 936-741 (SEQ ID NO: 1), 287-953 (SEQ ID NO: 2), 961c (SEQ ID NO: 3) or OMV (SEQ ID NO: 4), the method comprising the steps of:
   (a) providing one or more standard compositions wherein the one or more standard compositions comprise the same components at the same concentrations as the sample composition except that (i) the one or more standard compositions, as defined in clause 1, do not comprise the one or more MenB antigens present in the sample composition and instead comprise an antigen surrogate, in particular a MenB antigen surrogate, selected from ovalbumin, fosvitin, lactoferrin or alpha-casein and (ii) the one or more standard compositions comprise aluminium hydroxide at a known concentration,
   (b) obtaining one or more signals from the one or more standard compositions, wherein the level of the signals correlate with the concentration of aluminium hydroxide present in the standard compositions,
   (c) obtaining a signal from the sample composition in the same way as obtained for the one or more standard compositions in step (b) and
   (d) comparing the level of the signal from the sample composition with the level of the one or more signals from the standard compositions to establish the concentration of aluminium hydroxide in the sample composition.
6. A sample composition comprising aluminium hydroxide and one or more MenB antigens wherein the concentration of aluminium hydroxide in the composition was measured by the method of clause 5.
7. A sample composition comprising aluminium hydroxide and one or more MenB antigens wherein the concentration of aluminium hydroxide in the composition was measured using one or more standard compositions comprising an antigen surrogate selected from ovalbumin, fosvitin, lactoferrin or alpha-casein as a surrogate for the one or more MenB antigens, wherein the one or more standard compositions are analogous to the sample composition.
8. A composition comprising aluminium hydroxide and one or more MenB antigens wherein the composition was produced in the same batch as the sample composition of either clause 6 or 7.
9. The composition or method of clause 5 or 6 wherein the signals are established using electromagnetic spectroscopy.
10. The composition or method according to clause 9 wherein the electromagnetic spectroscopy is spectrophotometry.
11. The composition or method of any one of clauses 9 to 10 wherein the one or more signals are absorbance, transmittance or reflectance.
12. The composition or method according to clause 11 wherein the one or more signals are absorbance.
13. The composition or method of any one of clauses 9 to 10 wherein the electromagnetic radiation has a wavelength of about 1 to about 3,000 nm.
14. The composition or method according to clause 13 wherein the electromagnetic radiation has a wavelength of about 300 to about 1,000 nm.
15. The composition or method of clause 14 wherein the electromagnetic radiation has a wavelength of about 350 to about 800 nm.
16. The composition or method of clause 15 wherein the electromagnetic radiation has a wavelength of about 380 to about 600 nm.
17. The composition or method of clause 16 wherein the electromagnetic radiation has a wavelength of about 390 to about 500 nm.
18. The composition or method of clause 17 wherein the electromagnetic radiation has a wavelength of about 395 to about 450 nm.
19. The composition or method of clause 18 wherein the electromagnetic radiation has a wavelength of about 400 nm.
20. The composition or method of any one of clauses 9 to 19 wherein the electromagnetic spectroscopy is performed at about 10 to about 30° C.
21. The composition or method of clause 20 wherein the electromagnetic spectroscopy is performed at about 20° C.
22. The composition or method of any one of clauses 9 to 21 wherein the electromagnetic spectroscopy is performed shortly, such as immediately, after shaking the compositions for about 1 to about 5 minutes at about 500 to about 2,000 rpm.
23. The composition or method of any one of clauses 5, 6 and 9 to 22 wherein at least 2 signals are obtained from each of at least 2 standard compositions, wherein each of the standard compositions has a different concentration of aluminium hydroxide.
24. The composition or method of clause 23 wherein at least 3 signals are obtained from each of at least 3 standard compositions, wherein each of the standard compositions has a different concentration of aluminium hydroxide.
25. The composition or method of clause 24 wherein at least 4 signals are obtained from each of at least 4 standard compositions, wherein each of the standard compositions has a different concentration of aluminium hydroxide.
26. The composition or method of clause 25 wherein at least 5 signals are obtained from each of at least 5 standard compositions, wherein each of the standard compositions has a different concentration of aluminium hydroxide.
27. The composition, kit or method according to any one of clauses 1 to 26 wherein the concentration of aluminium hydroxide in the one or more standard compositions is about 0.1 to about 20 mg/ml.
28. The composition, use, kit or method according to clause 27 wherein the concentration of aluminium hydroxide in the one or more standard compositions is about 0.5 to about 10 mg/ml.
29. The composition, use, kit or method according to clause 28 wherein the concentration of aluminium hydroxide in the one or more standard compositions is about 0.7 to about 7 mg/ml.
30. The composition, use, kit or method according to clause 29 wherein the concentration of aluminium hydroxide in the one or more standard compositions is about 1.5 to about 4 mg/ml.
31. The composition, use, kit or method according to any one of clauses 2 to 30 wherein 3 or more standard compositions are used and the concentration of aluminium hydroxide in the standard compositions is around about 2 mg/ml, around about 3 mg/ml and around about 4 mg/ml.
32. The composition, use, kit or method according to clause 31 wherein 5 or more standard compositions are used and the concentration of aluminium hydroxide in the standard compositions is around about 2 mg/ml, around about 2.5 mg/ml, around about 3 mg/ml, around about 3.5 mg/ml and around about 4 mg/ml.
33. The composition or method of any one of clauses 5, 6 and 9 to 31 wherein a standard curve is produced from the one or more signals from the one or more standard compositions and wherein the level of the signal from the sample composition is compared with the standard curve to establish the concentration of aluminium hydroxide in the sample composition.
34. The composition, use, kit or method according to any one of clauses 5, 6 and 9 to 33 wherein the method consists essentially of the recited steps.
35. The composition, use, kit or method according to clause 34 wherein the method consists of the recited steps.
36. The composition, use, kit or method according to any one of clauses 2 to 35 wherein the concentration of aluminium hydroxide in the one or more standard compositions is +/−100% around the suspected concentration of aluminium hydroxide in the sample composition.
37. The composition, use, kit or method according to clause 36 wherein the concentration of aluminium hydroxide in the one or more standard compositions is +/−50% around the intended concentration of aluminium hydroxide in the sample composition.
38. The composition, use, kit or method according to clause 37 wherein the concentration of aluminium hydroxide in the sample composition is intended to be about 1 to about 5 mg/ml.
39. The composition, use, kit or method according to clause 38 wherein the concentration of aluminium hydroxide in the sample composition is intended to be around about 3 mg/ml.
40. The composition, use, kit or method according to any one of clauses 2 to 39 wherein the volume of the sample composition is about 0.1 to about 5.0 ml.
41. The composition, use, kit or method according to clause 40 wherein the volume of the sample composition is about 0.3 to about 3.0 ml.
42. The composition, use, kit or method according to any one of clauses 2 to 41 wherein the sample composition further comprises sodium chloride.
43. The composition, use, kit or method according to clause 42 wherein the sodium chloride is present at a concentration of about 1 to about 10 mg/ml.
44. The composition, use, kit or method according to clause 43 wherein the sodium chloride is present at a concentration of about 4 to about 8 mg/ml.
45. The composition, use, kit or method according to clause 44 wherein the sodium chloride is present at a concentration of about 6.25 mg/ml.
46. The composition, use, kit or method according to any one of clauses 4 to 45 wherein the sample composition further comprises histidine.
47. The composition, use, kit or method according to clause 46 wherein the histidine is present at a concentration of about 1 to about 20 mM.
48. The composition, use, kit or method according to clause 47 wherein the histidine is present at a concentration of about 5 to about 15 mM.
49. The composition, use, kit or method according to clause 48 wherein the histidine is present at a concentration of about 10 mM.
50. The composition, use, kit or method according to any one of clauses 2 to 49 wherein the sample composition further comprises sucrose.
51. The composition, use, kit or method according to clause 50 wherein the sucrose is present at a concentration of about 0.1 to about 10% w/v.
52. The composition, use, kit or method according to clause 51 wherein the sucrose is present at a concentration of about 1 to about 3% w/v.
53. The composition, use, kit or method according to clause 52 wherein the sucrose is present at a concentration of about 2.0% w/v.
54. The composition, use, kit or method according to any one of clauses 2 to 53 wherein the MenB antigen is selected in a group consisting of a polypeptide sharing at least 90%, such as 95%, such as 99%, such as 100% identity with SEQ ID NO: 1 to SEQ ID NO: 4, and SEQ ID NO: 9 to SEQ ID NO: 21.
55. The composition, use, kit or method according to any one of clauses 2 to 54 wherein the MenB antigen 936-741 is present in the sample composition.
56. The composition, use, kit or method according to clause 55 wherein MenB antigen 936-741 is present at a concentration of about 50 to about 150 mcg/ml.
57. The composition, use, kit or method according to clause 56 wherein MenB antigen 936-741 is present at a concentration of about 75 to about 125 mcg/ml.
58. The composition, use, kit or method according to clause 57 wherein MenB antigen 936-741 is present at a concentration of about 100 mcg/ml.
59. The composition, use, kit or method according to any one of clauses 54 to 58 wherein MenB antigen 936-741 comprises a polypeptide sharing at least about 90% identity with SEQ ID NO: 1.
60. The composition, use, kit or method according to clause 59 wherein MenB antigen 936-741 consists of a polypeptide sharing at least about 90% identity with SEQ ID NO: 1.
61. The composition, use, kit or method according to clause 59 wherein MenB antigen 936-741 comprises a polypeptide sharing at least about 95% identity with SEQ ID NO: 1.
62. The composition, use, kit or method according to clause 60 wherein MenB antigen 936-741 consists of a polypeptide sharing at least about 95% identity with SEQ ID NO: 1.
63. The composition, use, kit or method according to clause 61 wherein MenB antigen 936-741 comprises a polypeptide sharing at least about 99% identity with SEQ ID NO: 1.
64. The composition, use, kit or method according to clause 62 wherein MenB antigen 936-741 consists of a polypeptide sharing at least about 99% identity with SEQ ID NO: 1.
65. The composition, use, kit or method according to clause 63 wherein MenB antigen 936-741 comprises SEQ ID NO: 1.
66. The composition, use, kit or method according to clause 64 wherein MenB antigen 936-741 consists of SEQ ID NO: 1.
67. The composition, use, kit or method according to any one of clauses 2 to 66 wherein the MenB antigen 287-953 is present in the sample composition.
68. The composition, use, kit or method according to clause 67 wherein MenB antigen 287-953 is present at a concentration of about 50 to about 150 mcg/ml.
69. The composition, use, kit or method according to clause 68 wherein MenB antigen 287-953 is present at a concentration of about 75 to about 125 mcg/ml.
70. The composition, use, kit or method according to clause 69 wherein MenB antigen 287-953 is present at a concentration of about 100 mcg/ml.
71. The composition, use, kit or method according to any one of clauses 67 to 70 wherein MenB antigen 287-953 comprises a polypeptide sharing at least about 90% identity with SEQ ID NO: 2.
72. The composition, use, kit or method according to clause 71 wherein MenB antigen 287-953 consists of a polypeptide sharing at least about 90% identity with SEQ ID NO: 2.
73. The composition, use, kit or method according to clause 71 wherein MenB antigen 287-953 comprises a polypeptide sharing at least about 95% identity with SEQ ID NO: 2.
74. The composition, use, kit or method according clause 72 wherein MenB antigen 287-953 consists of a polypeptide sharing at least about 95% identity with SEQ ID NO: 2.
75. The composition, use, kit or method according to clause 73 wherein MenB antigen 287-953 comprises a polypeptide sharing at least about 99% identity with SEQ ID NO: 2.
76. The composition, use, kit or method according to clause 74 wherein MenB antigen 287-953 consists of a polypeptide sharing at least about 99% identity with SEQ ID NO: 2.
77. The composition, use, kit or method according to clause 75 wherein MenB antigen 287-953 comprises SEQ ID NO: 2.
78. The composition, use, kit or method according to clause 76 wherein MenB antigen 287-953 consists of SEQ ID NO: 2.
79. The composition, use, kit or method according to any one of clauses 2 to 78 wherein the MenB antigen 961c is present in the sample composition.
80. The composition, use, kit or method according to clause 79 wherein MenB antigen 961c is present at a concentration of about 50 to about 150 mcg/ml.
81. The composition, use, kit or method according to clause 80 wherein MenB antigen 961c is present at a concentration of about 75 to about 125 mcg/ml.
82. The composition, use, kit or method according to clause 81 wherein MenB antigen 961c is present at a concentration of about 100 mcg/ml.
83. The composition, use, kit or method according to any one of clauses 79 to 82 wherein MenB antigen 961c comprises a polypeptide sharing at least about 90% identity with SEQ ID NO: 3.
84. The composition, use, kit or method according to clause 83 wherein MenB antigen 961c consists of a polypeptide sharing at least about 90% identity with SEQ ID NO: 3.
85. The composition, use, kit or method according to clause 83 wherein MenB antigen 961c comprises a polypeptide sharing at least about 95% identity with SEQ ID NO: 3.
86. The composition, use, kit or method according to clause 84 wherein MenB antigen 961c consists of a polypeptide sharing at least about 95% identity with SEQ ID NO: 3.
87. The composition, use, kit or method according to clause 85 wherein MenB antigen 961c comprises a polypeptide sharing at least about 99% identity with SEQ ID NO: 3.
88. The composition, use, kit or method according to clause 86 wherein MenB antigen 961c consists of a polypeptide sharing at least about 99% identity with SEQ ID NO: 3.
89. The composition, use, kit or method according to clause 87 wherein MenB antigen 961c comprises SEQ ID NO: 3.
90. The composition, use, kit or method according to clause 88 wherein MenB antigen 961c consists of SEQ ID NO: 3.
91. The composition, use, kit or method according to any one of clauses 2 to 90 wherein the MenB antigen OMV is present in the sample composition.
92. The composition, use, kit or method according to clause 91 wherein MenB antigen OMV is present at a concentration of about 20 to about 80 mcg/ml.
93. The composition, use, kit or method according to clause 92 wherein MenB antigen OMV is present at a concentration of about 40 to about 60 mcg/ml.
94. The composition, use, kit or method according clause 93 wherein MenB antigen OMV is present at a concentration of about 50 mcg/ml.
95. The composition, use, kit or method according to any one of clauses 91 to 94 wherein MenB antigen OMV comprises a polypeptide sharing at least about 90% identity with SEQ ID NO: 4.
96. The composition, use, kit or method according to clause 95 wherein MenB antigen OMV consists of a polypeptide sharing at least about 90% identity with SEQ ID NO: 4.
97. The composition, use, kit or method according to clause 95 wherein MenB antigen OMV comprises a polypeptide sharing at least about 95% identity with SEQ ID NO: 4.
98. The composition, use, kit or method according to clause 96 wherein MenB antigen OMV consists of a polypeptide sharing at least about 95% identity with SEQ ID NO: 4.
99. The composition, use, kit or method according to clause 97 wherein MenB antigen OMV comprises a polypeptide sharing at least about 99% identity with SEQ ID NO: 4.
100. The composition, use, kit or method according to clause 98 wherein MenB antigen OMV consists of a polypeptide sharing at least about 99% identity with SEQ ID NO: 4.
101. The composition, use, kit or method according to clause 99 wherein MenB antigen OMV comprises SEQ ID NO: 4.
102. The composition, use, kit or method according to clause 100 wherein MenB antigen OMV consists of SEQ ID NO: 4.
103. The composition, use, kit or method according to any one of clauses 2 to 102 wherein all of MenB antigens 936-741, 287-953, 961c and OMV are present.
104. The composition, use, kit or method according to any one of clauses 2 to 103 wherein the sample composition consists essentially of one or more of MenB antigens 936-741, 287-953, 961c or OMV, aluminium hydroxide, sodium chloride, histidine and sucrose.
105. The composition, use, kit or method according to clause 104 wherein the sample composition consists of one or more of MenB antigens 936-741, 287-953, 961c or OMV, aluminium hydroxide, sodium chloride, histidine and sucrose.
106. The composition, use, kit or method of any one of clauses 2 to 105 wherein the concentration of the antigen surrogate in the one or more standard compositions is within about +/−10% of the concentration of the one or more MenB antigens in the sample composition.
107. The composition, use, kit or method of clause 106 wherein the concentration of the antigen surrogate in the one or more standard compositions is within about +/−5% of the concentration of the one or more MenB antigens in the sample composition.
108. The composition, use, kit or method of clause 107 wherein the concentration of the antigen surrogate in the one or more standard compositions is within about +/−1% of the concentration of the one or more MenB antigens in the sample composition.
109. The composition, use, kit or method of clause 108 wherein the concentration of the antigen surrogate in the one or more standard compositions is within about +/−0.1% of the concentration of the one or more MenB antigens in the sample composition.
110. The composition, use, kit or method of clause 109 wherein the concentration of the antigen surrogate in the one or more standard compositions is within about +/−0.05% of the concentration of the one or more MenB antigens in the sample composition.
111. The composition, use, kit or method of any one of clauses 2 to 110 wherein the concentration of the antigen surrogate in the one or more standard compositions is substantially the same as the concentration of all MenB antigens in the sample composition.
112. The composition, use, kit or method of clause 111 wherein the concentration of the antigen surrogate in the one or more standard compositions is the same as the concentration of all MenB antigens in the sample composition.
113. The composition, use, kit or method according to any one of clauses 1 to 112 wherein the concentration of the antigen surrogate is greater than about 50 mcg/ml.
114. The composition, use, kit or method according to clause 113 wherein the concentration of the antigen surrogate is greater than about 200 mcg/ml.
115. The composition, use, kit or method according to clause 114 wherein the concentration of the antigen surrogate is about 200 to about 500 mcg/ml.
116. The composition, use, kit or method according to clause 115 wherein the concentration of the antigen surrogate is about 250 mcg/ml.
117. The composition, use, kit or method of any one of clauses 1 to 116 wherein the antigen surrogate, in particular the MenB antigen surrogate, is ovalbumin.
118. The composition, use, kit or method of clause 117 wherein the ovalbumin is chicken ovalbumin.
119. The composition, use, kit or method of clause 118 wherein the ovalbumin is subclass A1 chicken ovalbumin.
120. The composition, use, kit or method of any one of clauses 117 to 119 wherein the ovalbumin is about 300 to about 450 amino acids in length.
121. The composition, use, kit or method of clause 120 wherein the ovalbumin is about 385 amino acids in length.
122. The composition, use, kit or method of any one of clauses 1 to 116 wherein the antigen surrogate, in particular the MenB antigen surrogate, is alpha-casein.
123. The composition, use, kit or method of clause 122 wherein the alpha-casein is bovine alpha-casein.
124. The composition, use, kit or method of clause 123 wherein the bovine alpha-casein is alpha-s1 bovine alpha-casein.
125. The composition, use, kit or method of clause 124 wherein the alpha-casein is the major form of alpha-s1 bovine alpha-casein.
126. The composition, use, kit or method of any one of clauses 1 to 116 wherein the antigen surrogate, in particular the MenB antigen surrogate, is fosvitin.
127. The composition, use, kit or method of clause 126 wherein the fosvitin is about 200 to about 250 amino acids in length.
128. The composition, use, kit or method of clause 120 wherein the fosvitin is about 217 amino acids in length.
129. The composition, use, kit or method of any one of clauses 1 to 116 wherein the antigen surrogate, in particular the MenB antigen surrogate, is lactoferrin.
130. The composition, use, kit or method of clause 126 wherein the lactoferrin is about 600 to about 800 amino acids in length.
131. The composition, use, kit or method of clause 120 wherein the lactoferrin is about 700 amino acids in length.
132. The composition, use, kit or method of any one of clauses 1 to 131 wherein at least one of the serine residues in the antigen surrogate, in particular the MenB antigen surrogate, is phosphorylated.
133. The composition, use, kit or method of clause 132 wherein at least half of the serine residues in the antigen surrogate, in particular the MenB antigen surrogate, are phosphorylated.
134. The composition, use, kit or method of clause 133 wherein all serine residues in the antigen surrogate, in particular the MenB antigen surrogate, are phosphorylated.
135. The composition, use, kit or method according to any one of clauses 1 to 134 wherein the standard composition further comprises sodium chloride.
136. The composition, use, kit or method according to clause 135 wherein the sodium chloride is present at a concentration of about 1 to about 10 mg/ml.
137. The composition, use, kit or method according to clause 136 wherein the sodium chloride is present at a concentration of about 4 to about 8 mg/ml.
138. The composition, use, kit or method according to clause 137 wherein the sodium chloride is present at a concentration of about 6.25 mg/ml.
139. The composition, use, kit or method according to any one of clauses 1 to 138 wherein the one or more standard compositions further comprises histidine.
140. The composition, use, kit or method according to clause 139 wherein the histidine is present at a concentration of about 1 to about 20 mM.
141. The composition, use, kit or method according to clause 140 wherein the histidine is present at a concentration of about 5 to about 15 mM.
142. The composition, use, kit or method according to clause 141 wherein the histidine is present at a concentration of about 10 mM.
143. The composition, use, kit or method according to any one of clauses 1 to 142 wherein the one or more standard compositions further comprise sucrose.
144. The composition, use, kit or method according to clause 143 wherein the sucrose is present at a concentration of about 0.1 to about 10% w/v.
145. The composition, use, kit or method according to clause 144 wherein the sucrose is present at a concentration of about 1 to about 3% w/V.
146. The composition, use, kit or method according to clause 145 wherein the sucrose is present at a concentration of about 2.0% w/v.
147. The composition, use, kit or method according to any one of clauses 1 to 146 wherein the one or more standard compositions comprise sodium chloride, histidine and sucrose.
148. The composition, use, kit or method according to clause 147 wherein the one or more standard compositions consist essentially of an antigen surrogate, in particular the MenB antigen surrogate, aluminium hydroxide, sodium chloride, histidine and sucrose.
149. The composition, use, kit or method according to clause 148 wherein the one or more standard compositions consist of an antigen surrogate, in particular the MenB antigen surrogate, aluminium hydroxide, sodium chloride, histidine and sucrose.
150. The composition, use, kit or method according to any one of clauses 1 to 149 wherein the one or more standard compositions do not comprise any MenB antigens.
151. The composition, use, kit or method according to clause 150 wherein the one or more standard compositions do not comprise any proteins other than the antigen surrogate, in particular the MenB antigen surrogate.
152. The composition, use, kit or method according to any one of clauses 1 to 151 wherein the volume of the one or more standard compositions is about 0.1 to about 5.0 ml.
153. The composition, use, kit or method according to clause 152 wherein the volume of the one or more standard compositions is about 0.3 to about 3.0 ml.

### Examples

### Materials

Men B vaccines used in the examples below comprised the components set out in Table 4 below.

TABLE 4

| Component | Concentration | |
|---|---|---|
| Al(OH)₃ | 3 mg/ml | |
| NaCl | 6.25 | mg/ml |
| Histidine pH 6.3 | 10 | mM |
| 936-741 | 100 | mcg/ml |
| 961c | 100 | mcg/ml |
| 287-953 | 100 | mcg/ml |
| OMV | 50 | mcg/ml |
| Sucrose | 2.0% | w/v |

The antigens were adsorbed (>90%) to the aluminium hydroxide adjuvant. The pH of the vaccines was maintained at 6.5 (±0.5) and the osmolarity of the vaccines was maintained at around 0.300 Osmol/Kg

### Example 1—Measuring Aluminium Hydroxide Concentration Using Established Methodology

A total of 700 MenB vaccine-containing pre-filled syringes (PFS) distributed across 7 nests (multiple syringe containers) were produced. For each nest, the first and last PFS (14 PFS in total, also referred to as ‘sample compositions’) were analyzed in duplicate (twice per PFS) by manually performing the established method. This method was as follows.

Six standard compositions were prepared wherein each standard composition consisted of the components set out in Table 4 above, but wherein the aluminium hydroxide concentration in each of the six standard compositions was instead as set out in Table 5 below.

TABLE 5

| Standard Composition | Aluminium Hydroxide Concentration (mg/ml) |
|---|---|
| 1 | 3.6 |
| 2 | 3.3 |
| 3 | 2.7 |
| 4 | 2.4 |
| 5 | 2.0 |
| 6 | 1.0 |

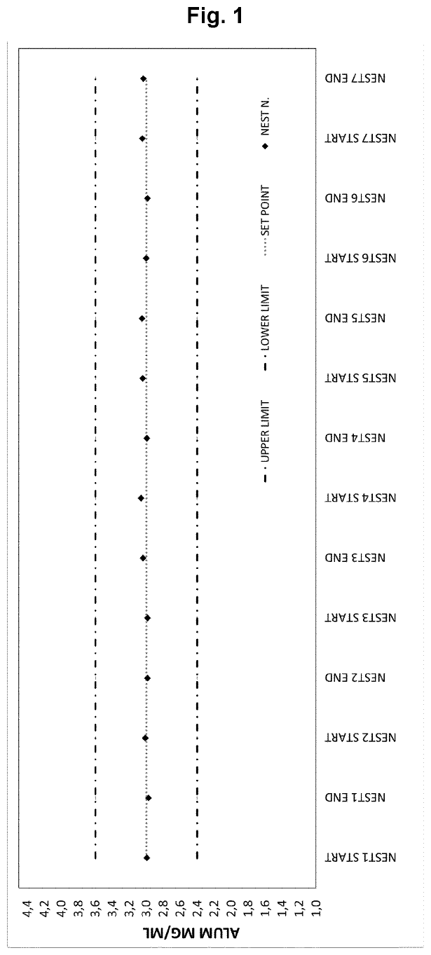

The standard compositions and sample compositions were loaded manually onto a 96 well plate. Measurements were taken from the standard compositions to produce a standard curve. Measurements were taken from the sample compositions and the results were compared to the standard curve to measure the concentration of aluminium hydroxide in the sample compositions. The measurements are shown in FIG. 1. As expected, all sample compositions were found to contain aluminium hydroxide at concentrations of close to 3.0 mg/ml.

An analogous experiment was carried out using a robotic liquid handling device. Similar results were obtained.

### Example 2—Spectrophotometry to Establish Aluminium Hydroxide Concentration by Comparison to a Standard Curve

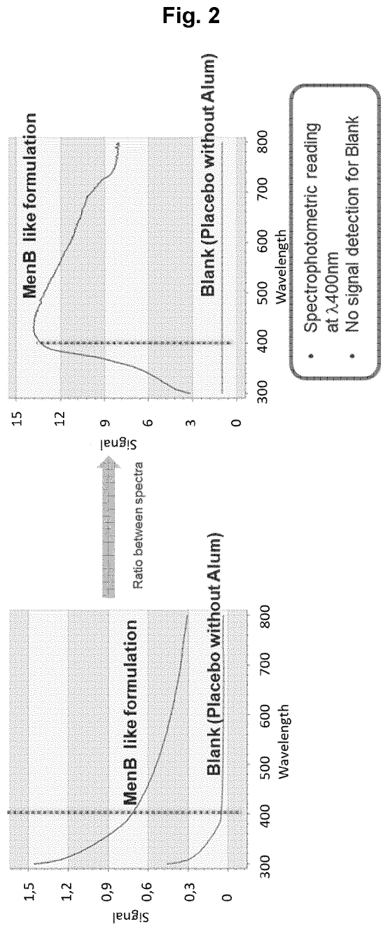

A spectrophotometric method was developed to establish the concentration of aluminium hydroxide in a sample composition, using a standard curve. Since aluminium hydroxide is an opalescent suspension and because of the absence of a single clear absorption peak, the ratio between the aluminium suspension spectra and its blank allows the identification of the wavelength at which the optical signal of the selected sample shows the higher significative response compared to its blank within the visible range. Through the analysis of several spectra, 400 nm was selected as the optimal wavelength to perform a specific quantification of aluminium hydroxide because it represents the maximum distance between the sample and its respective blank. An example of raw spectra for a MenB like formulation and the resulting curve showing the ratio between spectra is shown in FIG. 2. The final values are expressed as absorbance (log 1/T) and the signal is proportional to the aluminium hydroxide content.

### Example 3—Identification of a Surrogate Protein for MenB Antigens

Various proteins were tested for their suitability as substitutes for MenB antigens in standard compositions such as those detailed in Example 1 above. These proteins are listed in Table 6 below.

TABLE 6

| | | | | Phosphorylated |
|---|---|---|---|---|
| | | | | (if yes, number |
| | | Molecular | Amino | of potentially |
| | | weight | acid | phosphorylated |
| Test protein | IP | (kDa) | residues | groups) |
| Ovalbumin | 4.5 | 42.7 | 385 | Yes (1-2) |
| BSA | 4.7 | 66.5 | 583 | No |
| Lysozyme (from | 10.5-11.0 | 14.6 | 129 | No |
| chicken egg) | | | | |
| Alpha-casein | 4.2-4.8 | 22.0-25.0 | 195 | Yes (1-8) |
| Dephosphorylated | 4.6 | 25 | 195 | No |
| alpha-casein | | | | |
| Fosvitin | 4 | 35 | 217 | Yes (~80) |
| Alpha-lactoalbumin | 4.2-4.5 | 14.2 | 123 | No |
| Bovine | 6.8 | 64.5 | 574 | No |
| haemoglobin | | | | |
| Pepsin | 3.24 | 34.5 | 326 | No |
| Lactoferrin | 8.7 | 80 | 700 | No |

Standard curves were produced analogously to that set out in Example 1, but in this instance wherein each of the proteins in Table 6 above was used in place of the MenB antigens. To this end, ten sets of eight compositions were prepared, wherein the compositions in each set contained one of the proteins listed in Table 6 above at the same concentration as the MenB antigens used in Example 1, along with further components (as set out in Table 7 below), plus wherein the aluminium hydroxide concentration in each of the eight compositions per set was instead as set out in Table 8 below.

TABLE 7

| Component | Concentration | |
|---|---|---|
| Al(OH)₃ | 1.5 to 4 mg/ml | |
| NaCl | 6.25 | mg/ml |
| Histidine pH 6.3 | 10 | mM |
| Test protein | 350 | mcg/ml |
| Sucrose | 2.0% | w/v |

TABLE 8

| Standard Composition | Aluminium Hydroxide Concentration (mg/ml) |
|---|---|
| 1 | 4.0 |
| 2 | 3.6 |
| 3 | 3.4 |
| 4 | 3.0 |
| 5 | 2.6 |
| 6 | 2.4 |
| 7 | 2.0 |
| 8 | 1.5 |

Comparison compositions comprising MenB antigens with different aluminium hydroxide concentrations were also produced. These consisted of the components set out in Table 9 below, at a series of aluminium hydroxide concentrations as set out in Table 8 above.

TABLE 9

| Component | Concentration | |
|---|---|---|
| Al(OH)₃ | 1.5 to 4 mg/ml | |
| NaCl | 6.25 | mg/ml |
| Histidine pH 6.3 | 10 | mM |
| 936-741 | 100 | mcg/ml |
| 961c | 100 | mcg/ml |
| 287-953 | 100 | mcg/ml |
| OMV | 50 | mcg/ml |
| Sucrose | 2.0% | w/v |

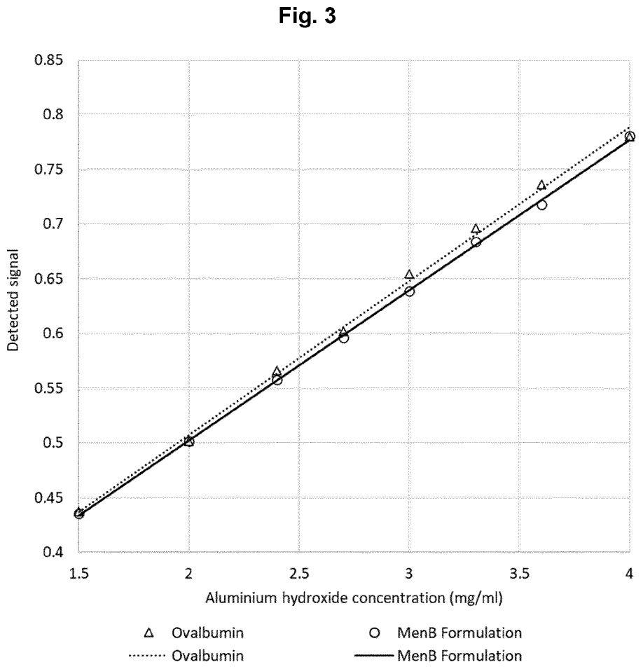

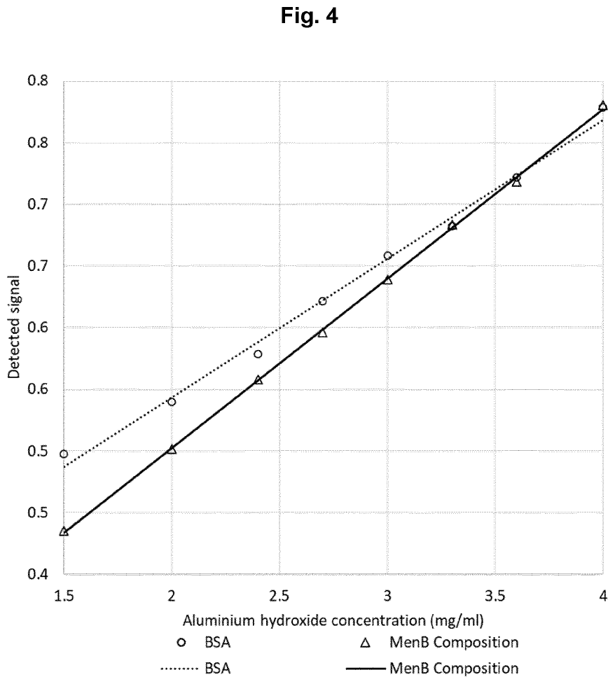

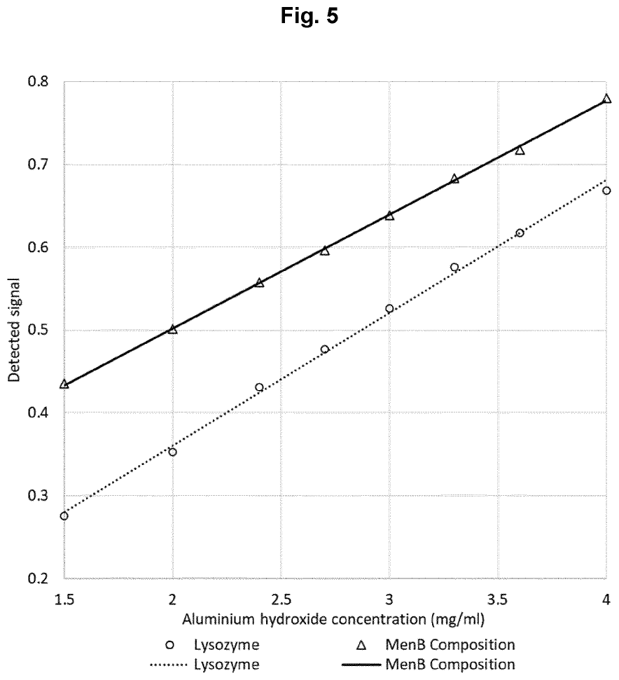

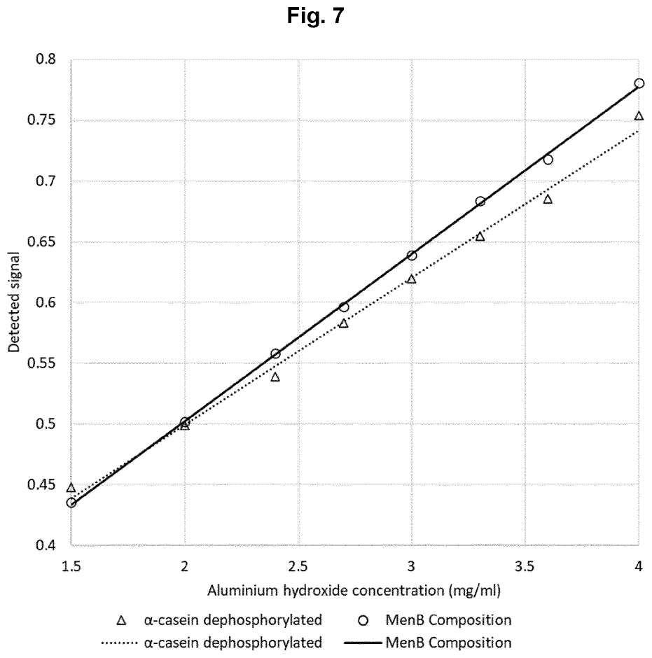

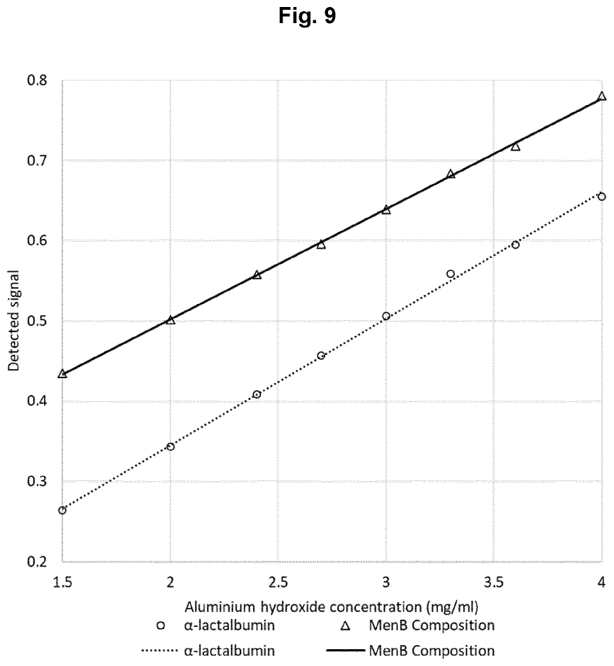

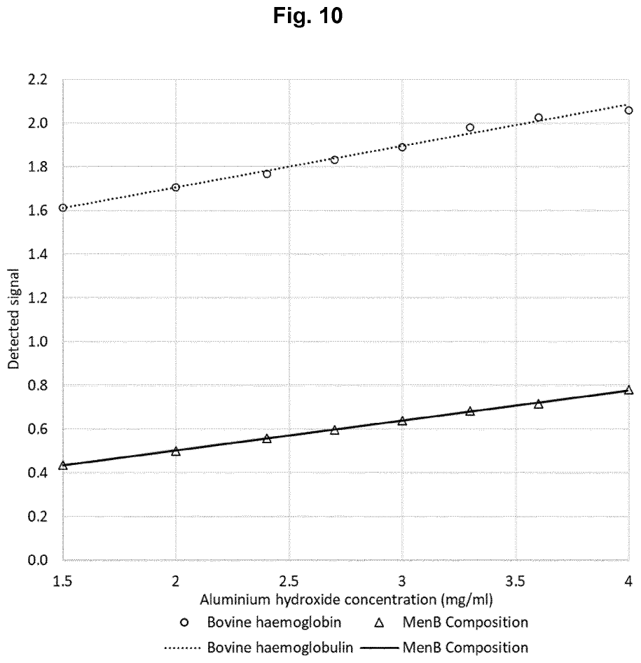

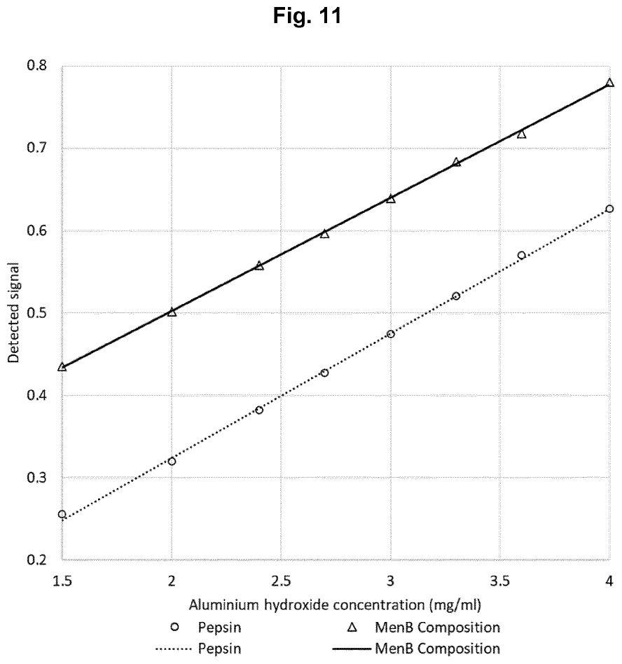

Spectrophotometric absorbance readings as detailed above in Example 2 were taken from the test protein compositions and compared with readings taken from the comparison compositions. The results are shown in FIGS. 3 to 11. The equations for the lines depicted in these figures are provided in Table 10 below, with the results from the comparison composition comprising MenB antigens underlined.

TABLE 10

| Protein | R² | Equation |
|---|---|---|
| Ovalbumin | 0.998 | Y = 0.1406 + 0.2265 |
| BSA | 0.994 | Y = 0.1126x + 0.3182 |
| Lysozyme (from chicken egg) | 0.997 | Y = 0.1602x + 0.0400 |
| Alpha-casein | 0.991 | Y = 0.1338x + 0.2446 |
| Dephosphorylated alpha-casein | 0.995 | Y = 0.1211x + 0.2570 |
| Fosvitin | 0.996 | Y = 0.1328x + 0.2705 |
| Alpha-lactoalbumin | 0.999 | Y = 0.1579x + 0.2940 |
| Bovine haemoglobin | 0.982 | Y = 0.1893x + 1.3284 |
| Pepsin | 0.999 | Y = 0.151x + 0.0221 |
| Lactoferrin | 0.999 | Y = 0.1436x + 0.3557 |
| Comparison composition | 0.999 | Y = 0.1374x + 0.2275 |
| containing MenB antigens | | |

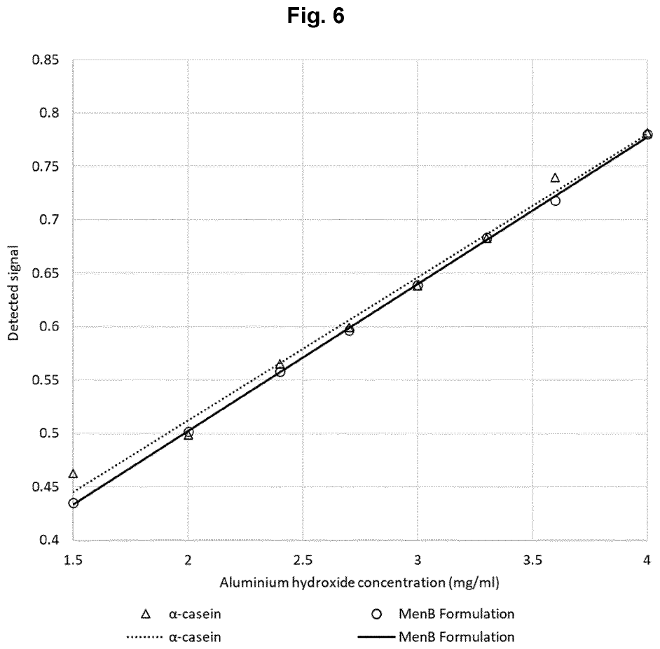

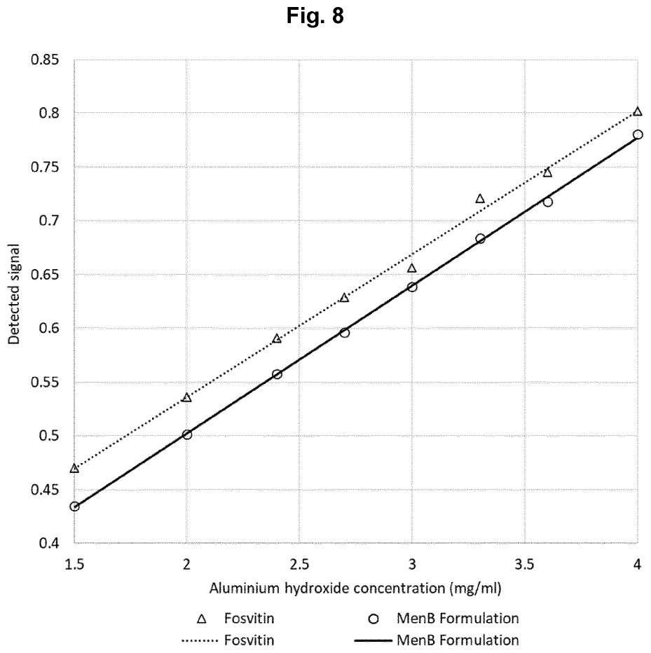

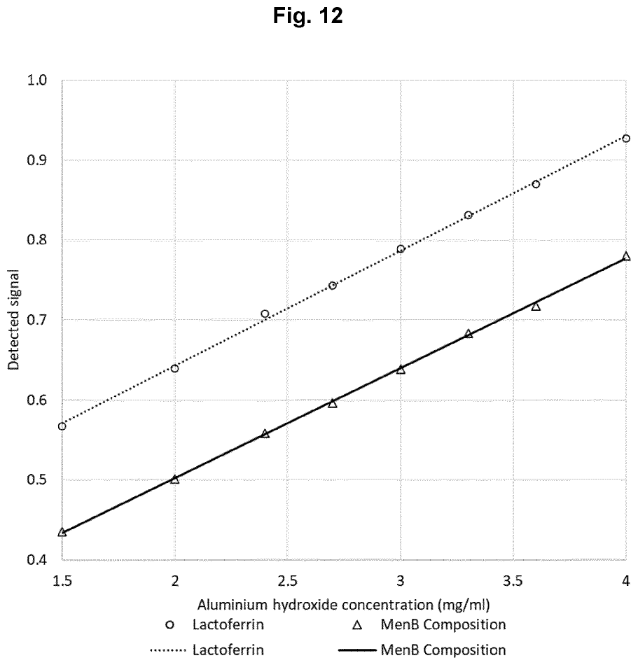

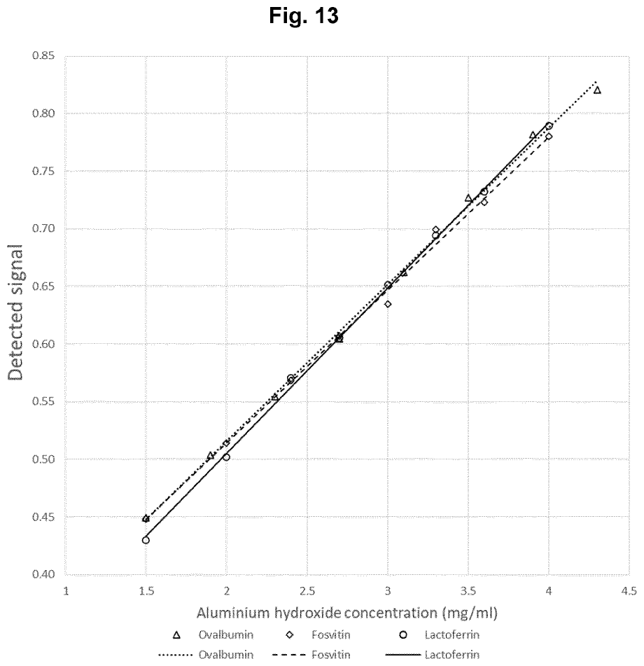

It may be noted that the equations for the lines produced for ovalbumin (FIG. 3), fosvitin (FIG. 8), lactoferrin (FIG. 12) and alpha-casein (FIG. 6) had very similar slope values to the line produced for the MenB comparison composition. Fosvitin and lactoferrin had distinct y-intercept values to the line produced for the MenB comparison composition, but by applying subtraction the curves overlaid closely (FIG. 13). It was considered that this may make ovalbumin, fosvitin, lacroferrin, and alpha-casein suitable surrogate proteins for MenB antigens. Ovalbumin displayed the most similar y-intercept and slope values and required no subtraction. Ovalbumin also benefitted from a high R2 value, adsorbed to the aluminium hydroxide quickly and was readily soluble in the buffer solution. Ovalbumin was therefore taken forward for further testing as a surrogate for MenB antigens.

### Example 4—Measuring Aluminium Hydroxide Concentration Using the Methods of the Invention

Sample compositions comprising MenB antigens and aluminium hydroxide were prepared as set out in Table 6 above.

To produce a standard curve, standard compositions comprising ovalbumin were prepared. The solutions used for producing the standard compositions were manually prepared starting from 1 mg/ml ovalbumin in KH₂PO₄ 10 mM, NaCl 150 mM at pH 7.00 as set out in Table 11 below.

TABLE 11

| Solution | Components and concentration |
|---|---|
| Stock solution | AlOH₃ 5 mg/ml; |
| | NaCl 6.25 mg/ml; |
| | Histidine pH 6.5 10 mM; |
| | Sucrose 2% w/v |
| | Ovalbumin 350 mcg/ml |
| Diluent Solution/Blank | NaCl 6.25 mg/ml; |
| | Histidine pH 6.5, 10 mM; |
| | Sucrose 2% w/v |
| | Ovalbumin 350 mcg/ml |

Starting with the two solutions shown in the Table 11, the liquid handling device was used to produce multiple standard compositions. The standard compositions (eight aluminium hydroxide concentrations, with two replicates each), sample Men B compositions (2 replicate wells per sample) and blank were loaded onto a plate (200 mcl/well). The standard compositions contained the components listed in Table 12 below, with aluminium hydroxide at the concentrations provided in Table 13 below.

TABLE 12

| Component | Concentration | |
|---|---|---|
| Al(OH)₃ | 1.5 to 4.3 mg/ml | |
| NaCl | 6.25 | mg/ml |
| Histidine pH 6.3 | 10 | mM |
| Ovalbumin | 350 | mcg/ml |
| Sucrose | 2.0% | w/v |

TABLE 13

| Standard Composition | Aluminium Hydroxide Concentration (mg/ml) |
|---|---|
| 1 | 4.3 |
| 2 | 3.9 |
| 3 | 3.5 |
| 4 | 3.2 |
| 5 | 2.7 |
| 6 | 2.3 |
| 7 | 1.9 |
| 8 | 1.5 |

The plate was transferred into a spectrophotometer. After shaking at 1200 rpm for one minute and maintaining a plate temperature of 20° C., absorbance readings were taken at 400 nm. By comparing the readings from the sample compositions with the standard curve produced from the standard compositions, the concentration of aluminium hydroxide in the sample compositions was measured as 3.0 mg/ml, as expected. The method was therefore successful.

### Example 5—Analysing the Reliability of the Methods of the Invention when Applied to Varied Aluminium Hydroxide Concentrations

The methods of the invention were analysed further using varied concentrations of aluminium hydroxide in the sample compositions. The analysis was performed in 12 analytical sessions performed by three operators. In each session, two replicates of the following sample compositions were tested (Table 14, below).

TABLE 14

| Sample Composition | Aluminium Hydroxide Concentration (mg/ml) |
|---|---|
| 1 | 2.0 |
| 2 | 2.5 |
| 3 | 3.0 |
| 4 | 3.5 |
| 5 | 4.0 |

The analysis was performed according to the scheme provided in Table 15 below.

TABLE 15

| Oper- | Ovalbumin | Plate | | | | | | | |
|---|---|---|---|---|---|---|---|---|---|
| ator | lot | lot | Session | Replicate | Sample composition | | | | |
| 1 | 1 | 1 | 1 | 1 | 1 | 2 | 3 | 4 | 5 |
| | | | | 2 | 1 | 2 | 3 | 4 | 5 |
| | 2 | 2 | 2 | 1 | 1 | 2 | 3 | 4 | 5 |
| | | | | 2 | 1 | 2 | 3 | 4 | 5 |
| | 1 | 1 | 3 | 1 | 1 | 2 | 3 | 4 | 5 |
| | | | | 2 | 1 | 2 | 3 | 4 | 5 |
| | 2 | 2 | 4 | 1 | 1 | 2 | 3 | 4 | 5 |
| | | | | 2 | 1 | 2 | 3 | 4 | 5 |
| 2 | 1 | 1 | 5 | 1 | 1 | 2 | 3 | 4 | 5 |
| | | | | 2 | 1 | 2 | 3 | 4 | 5 |
| | 2 | 2 | 6 | 1 | 1 | 2 | 3 | 4 | 5 |
| | | | | 2 | 1 | 2 | 3 | 4 | 5 |
| | 1 | 1 | 7 | 1 | 1 | 2 | 3 | 4 | 5 |
| | | | | 2 | 1 | 2 | 3 | 4 | 5 |
| | 2 | 2 | 8 | 1 | 1 | 2 | 3 | 4 | 5 |
| | | | | 2 | 1 | 2 | 3 | 4 | 5 |
| 3 | 1 | 1 | 9 | 1 | 1 | 2 | 3 | 4 | 5 |
| | | | | 2 | 1 | 2 | 3 | 4 | 5 |
| | 2 | 2 | 10 | 1 | 1 | 2 | 3 | 4 | 5 |
| | | | | 2 | 1 | 2 | 3 | 4 | 5 |
| | 1 | 1 | 11 | 1 | 1 | 2 | 3 | 4 | 5 |
| | | | | 2 | 1 | 2 | 3 | 4 | 5 |
| | 2 | 2 | 12 | 1 | 1 | 2 | 3 | 4 | 5 |
| | | | | 2 | 1 | 2 | 3 | 4 | 5 |

By applying the methodology described above in Example 4, the concentration of aluminium hydroxide in the sample compositions was measured. The measured concentrations for each sample are provided in Table 16 below.

TABLE 16

| | | | | Sample 1 | Sample 2 | Sample 3 | Sample 4 | Sample 5 |
|---|---|---|---|---|---|---|---|---|
| | Ovalbumin | Plate | | 2.0 mg/ml | 2.5 mg/ml | 3.0 mg/ml | 3.5 mg/ml | 4.0 mg/ml |
| Operator | lot | lot | Session | meas. conc. | meas. conc. | meas. conc. | meas. conc. | meas. conc. |
| 1 | 1 | 1 | 1 | 2.03339 | 2.48727 | 2.96411 | 3.56374 | 4.04019 |
| | | | | 1.99434 | 2.44434 | 2.95452 | 3.53582 | 3.97114 |
| | 2 | 2 | 2 | 2.14188 | 2.45276 | 3.16969 | 3.61712 | 4.00192 |
| | | | | 2.06439 | 2.47659 | 3.16701 | 3.51871 | 4.05953 |
| | 1 | 1 | 3 | 2.07027 | 2.56848 | 3.08525 | 3.48250 | 3.75229 |
| | | | | 2.03766 | 2.54250 | 2.97312 | 3.45684 | 3.83403 |
| | 2 | 2 | 4 | 2.06463 | 2.55874 | 3.04072 | 3.59992 | 4.06221 |
| | | | | 2.04624 | 2.57189 | 3.05579 | 3.59256 | 4.07080 |
| 2 | 1 | 1 | 5 | 2.08322 | 2.52810 | 3.15176 | 3.30509 | 3.91133 |
| | | | | 2.00398 | 2.47633 | 3.00439 | 3.37491 | 3.80801 |
| | 2 | 2 | 6 | 2.19055 | 2.41147 | 3.00613 | 3.46102 | 3.92578 |
| | | | | 1.83192 | 2.41655 | 2.99654 | 3.49433 | 3.99784 |
| | 1 | 1 | 7 | 2.07867 | 2.45742 | 3.16137 | 3.65721 | 4.20633 |
| | | | | 1.96999 | 2.53598 | 3.16177 | 3.64168 | 4.15156 |
| | 2 | 2 | 8 | 2.06552 | 2.56263 | 3.07046 | 3.40293 | 4.13996 |
| | | | | 2.05156 | 2.58650 | 3.00256 | 3.46172 | 3.99539 |
| 3 | 1 | 1 | 9 | 2.11692 | 2.67129 | 3.06703 | 3.52839 | 3.97403 |
| | | | | 2.12898 | 2.59750 | 3.04835 | 3.42864 | 4.06873 |
| | 2 | 2 | 10 | 2.08038 | 2.53846 | 3.16990 | 3.59814 | 4.14257 |
| | | | | 2.22194 | 2.59252 | 3.13750 | 3.56740 | 4.14409 |
| | 1 | 1 | 11 | 2.17900 | 2.63925 | 3.19944 | 3.67558 | 4.07169 |
| | | | | 2.14569 | 2.61865 | 3.17047 | 3.70560 | 4.06064 |
| | 2 | 2 | 12 | 2.14679 | 2.52967 | 3.13306 | 3.68839 | 3.89957 |
| | | | | 2.10019 | 2.53456 | 3.15830 | 3.49818 | 4.26084 |

It can be seen that the methods of the invention provided measurements of aluminium hydroxide concentration which were extremely close to the known concentrations. It should be noted that the range of aluminium hydroxide concentration variation in these samples is expected to be far greater than that which may occur during GMP production.

### Example 6—Analysing the Reliability of the Methods of the Invention when Applied to Varied Aluminium Hydroxide Concentrations and Varied MenB Antigen Concentrations

The methods of the invention were analysed further using (a) varied concentrations of aluminium hydroxide and also (b) varied concentrations of MenB antigens, in the sample compositions. The following samples were prepared:
5 samples having 5 different concentrations of aluminium hydroxide and containing 30% lower concentration of MenB antigens than the conventional target (3 mg/ml).
5 samples having 5 different concentrations of aluminium hydroxide and containing 30% higher concentration of MenB antigens than the conventional target (3 mg/ml).

The samples were prepared as set out in Table 17 below.

TABLE 17

| MenB antigens | | |
|---|---|---|
| concentration | | Aluminium hydroxide |
| (Δtarget) | Sample label | Concentration (mg/ml) |
| −30% | 1 LOW | 2 |
| −30% | 2 LOW | 2.5 |
| −30% | 3 LOW | 3 |
| −30% | 4 LOW | 3.5 |
| −30% | 5 LOW | 4 |
| +30% | 1 HIGH | 2 |
| +30% | 2 HIGH | 2.5 |
| +30% | 3 HIGH | 3 |
| +30% | 4 HIGH | 3.5 |
| +30% | 5 HIGH | 4 |

One replicate of each sample was included.

The analysis was performed by three operators. The results are provided in Table 18, below.

TABLE 18

| | | | | | | Sample 1 | Sample 2 | Sample 3 | Sample 4 | Sample 5 |
|---|---|---|---|---|---|---|---|---|---|---|
| | Ovalbumin | Plate | | | Antigen | 2.0 mg/ml | 2.5 mg/ml | 3.0 mg/ml | 3.5 mg/ml | 4.0 mg/ml |
| Operator | lot | lot | Session | Replicate | conc. | meas. conc. | meas. conc. | meas. conc. | meas. conc. | meas. conc. |
| 1 | 1 | 1 | 1 | 1 | Low | 1.7291441 | 2.230925 | 3.0874613 | 3.6754295 | 4.0928342 |
| 1 | 1 | 1 | 1 | 1 | High | 2.1396604 | 2.6210728 | 3.0678871 | 3.4973095 | 3.8925123 |
| 1 | 2 | 2 | 2 | 1 | Low | 1.8818391 | 2.4366172 | 3.1220263 | 3.3555972 | 3.8374521 |
| 1 | 2 | 2 | 2 | 1 | High | 2.1703645 | 2.623657 | 3.0806481 | 3.4842794 | 4.0062723 |
| 1 | 1 | 1 | 3 | 1 | Low | 1.9193331 | 2.4226444 | 2.8245675 | 3.5456994 | 3.8531076 |
| 1 | 1 | 1 | 3 | 1 | High | 2.1669143 | 2.6383659 | 3.0907571 | 3.4206223 | 3.8928393 |
| 1 | 2 | 2 | 4 | 1 | Low | 2.0170399 | 2.5505885 | 3.0491001 | 3.5379043 | 3.8099444 |
| 1 | 2 | 2 | 4 | 1 | High | 2.1427399 | 2.6202614 | 3.1489235 | 3.5450007 | 4.0287507 |
| 2 | 1 | 1 | 5 | 1 | Low | 1.8186549 | 2.2328002 | 3.0225484 | 3.3260083 | 3.9341147 |
| 2 | 1 | 1 | 5 | 1 | High | 2.0626261 | 2.5351852 | 2.9617754 | 3.4277735 | 3.8310094 |
| 2 | 2 | 2 | 6 | 1 | Low | 1.8237068 | 2.4536019 | 2.9041512 | 3.5776222 | 4.2472566 |
| 2 | 2 | 2 | 6 | 1 | High | 2.1129312 | 2.5994637 | 3.0038503 | 3.4824101 | 3.8360593 |
| 2 | 1 | 1 | 7 | 1 | Low | 1.8359346 | 2.4882275 | 2.9312545 | 3.5519889 | 4.2150191 |
| 2 | 1 | 1 | 7 | 1 | High | 2.0908475 | 2.587561 | 3.1070019 | 3.6243523 | 4.0516769 |
| 2 | 2 | 2 | 8 | 1 | Low | 1.8930343 | 2.4738697 | 2.9860801 | 3.6176995 | 4.034092 |
| 2 | 2 | 2 | 8 | 1 | High | 2.1362663 | 2.6259118 | 3.0597613 | 3.5537397 | 3.996063 |
| 3 | 1 | 1 | 9 | 1 | Low | 1.9964526 | 2.3746342 | 3.1340823 | 3.3787126 | 4.0324344 |
| 3 | 1 | 1 | 9 | 1 | High | 2.2372726 | 2.6957148 | 3.1650457 | 3.4945563 | 3.9066488 |
| 3 | 2 | 2 | 10 | 1 | Low | 2.064592 | 2.5037186 | 2.8389777 | 3.6546275 | 3.7563128 |
| 3 | 2 | 2 | 10 | 1 | High | 2.212199 | 2.8149898 | 3.2699102 | 3.2928672 | 4.0588105 |
| 3 | 1 | 1 | 11 | 1 | Low | 2.0351363 | 2.5496981 | 3.0663359 | 3.528651 | 3.9212547 |
| 3 | 1 | 1 | 11 | 1 | High | 2.2832194 | 2.7408728 | 3.2603032 | 3.3779838 | 4.0779873 |
| 3 | 2 | 2 | 12 | 1 | Low | 1.9789852 | 2.385649 | 2.8350911 | 3.5238288 | 4.1172912 |
| 3 | 2 | 2 | 12 | 1 | High | 2.1563963 | 2.5531756 | 3.2407002 | 3.3579823 | 3.9578939 |

It can be seen that the methods of the invention provided measurements of aluminium hydroxide concentration which were extremely close to the known concentrations. It should be noted that the range of aluminium hydroxide concentration variation and the range of antigen concentration variation in these samples is expected to be far greater than that which may occur during GMP production.

### REFERENCES

The references below are incorporated herein in their entirety.
HogenEsch, Harm et al. “Optimizing the utilization of aluminum adjuvants in vaccines: you might just get what you want” npj Vaccines 3, 51 (2018)
Tappero, Jordan W., et al. “Immunogenicity of 2 serogroup B outer-membrane protein meningococcal vaccines: a randomized controlled trial in Chile.” Jama 281.16 (1999): 1520-1527.
Vaccine Design (1995) eds. Powell & Newman. ISBN: 030644867X. Plenum
van der Voort, E. Rouppe, et al. “Specificity of human bactericidal antibodies against PorA P1. 7, 16 induced with a hexavalent meningococcal outer membrane vesicle vaccine.” Infection and immunity 64.7 (1996): 2745-2751.
WO01/52885 (OUTER MEMBRANE VESICLE (OMV) VACCINE COMPRISING N. MENINGITIDIS SEROGROUP B OUTER MEMBRANE PROTEINS).
WO03/007985 (CAPSULAR POLYSACCHARIDE SOLUBILISATION AND COMBINATION VACCINES).
WO2003/010194 (MENINGOCOCCUS ADHESINS).
WO2003/020756 (HYBRID AND TANDEM EXPRESSION OF NEISSERIAL PROTEINS).
WO2004/048404 (MULTIPLE VARIANTS OF MENINGOCOCCAL PROTEIN NMB1870).
WO2007/060548 (CHIMERIC, HYBRID AND TANDEM POLYPEPTIDES OF MENINGOCOCCAL NMB1870).
WO2010/109323 (ADJUVANTING MENINGOCOCCAL FACTOR H BINDING PROTEIN).
WO2012/032489 (NON-LIPIDATED VARIANTS OF NEISSERIA MENINGITIDIS ORF2086 ANTIGENS).
WO2015/128480 (MODIFIED MENINGOCOCCAL FHBP POLYPEPTIDES).
WO2016/008960 (MODIFIED MENINGOCOCCAL FHBP POLYPEPTIDES).
WO2016/008961 (MENINGOCOCCUS VACCINES).
WO2018/042017 (VACCINES FOR NEISSERIA GONORRHOEAE).
WO2020/030782 (MODIFIED MENINGOCOCCAL FHBP POLYPEPTIDES).
WO2020/165711 (NEISSERIA MENINGITIDISCOMPOSITIONS AND METHODS THEREOF).
WO2020/182635 (CARBOCYCLIC DERIVATIVES AND CONJUGATED DERIVATIVES THEREOF, AND THEIR USE IN VACCINES).

1-16. (canceled)

17. A standard composition comprising aluminium hydroxide and an antigen surrogate comprising ovalbumin, fosvitin, lactoferrin, or alpha-casein.

18. The composition of claim 17, wherein the antigen surrogate is a MenB antigen surrogate.

19. A method for measuring the concentration of aluminium hydroxide in a sample composition comprising the step of using a standard composition comprising an antigen surrogate for one or more MenB antigens, said surrogate comprising ovalbumin, fosvitin, lactoferrin or alpha-casein.

20. A method of measuring the concentration of aluminium hydroxide in a sample composition, wherein the sample composition comprises one or more MenB antigens having at least about 90% identity with any of SEQ ID NO: 1 to SEQ ID NO: 4, and SEQ ID NO: 9 to SEQ ID NO: 21, the method comprising:
(a) obtaining one or more signals from one or more standard compositions, the one or more standard compositions comprising the same components at the same concentrations as the sample composition except that:
(i) the one or more standard compositions do not comprise the one or more MenB antigens present in the sample composition but instead comprise an antigen surrogate comprising ovalbumin, fosvitin, lactoferrin, or alpha-casein, and
(ii) the one or more standard compositions comprise aluminium hydroxide at a known concentration, wherein the level of the signals correlate with the concentration of aluminium hydroxide present in the standard compositions,
(b) obtaining a signal from the sample composition in the same way as obtained for the one or more standard compositions as in (a), and
(c) comparing the signal from the sample composition with the one or more signals from the standard compositions to determine the concentration of aluminium hydroxide in the sample composition.

21. The method according to claim 20 wherein the signals are established using spectrophotometry.

22. The method of claim 21 wherein the electromagnetic radiation has a wavelength of about 350 to about 800 nm.

23. The method of claim 20 wherein at least 2 signals are obtained from each of at least 2 standard compositions, wherein each of the standard compositions has a different concentration of aluminium hydroxide.

24. The method of claim 20 wherein a standard curve is produced from the one or more signals from the one or more standard compositions and wherein the level of the signal from the sample composition is compared with the standard curve to establish the concentration of aluminium hydroxide in the sample composition.

25. The method of claim 20 wherein the sample composition comprises MenB antigens 936-741 (of SEQ ID NO: 1), 287-953 (of SEQ ID NO: 2), 961c (of SEQ ID NO: 3) and OMV (of SEQ ID NO: 4).

26. The composition of claim 17 wherein the antigen surrogate is ovalbumin or alpha-casein.

27. The composition of claim 17 wherein at least one of the serine residues in the antigen surrogate is phosphorylated.

28. The composition according to claim 17 wherein the one or more standard compositions consist of an antigen surrogate, aluminium hydroxide, sodium chloride, histidine and sucrose.

29. The composition according to claim 17 wherein the one or more standard compositions do not comprise any proteins other than the antigen surrogate.

30. The method according to claim 20 wherein the concentration of aluminium hydroxide in the sample composition is about 1 to about 5 mg/ml.

31. The method according to claim 20 wherein the sample composition comprises MenB antigens 936-741 (of SEQ ID NO: 1), 287-953 (of SEQ ID NO: 2), 961c (of SEQ ID NO: 3) or OMV (of SEQ ID NO: 4).

32. The method of claim 20 wherein the antigen surrogate is ovalbumin or alpha-casein.

33. The method of claim 20 wherein at least one of the serine residues in the antigen surrogate is phosphorylated.

34. The method according to claim 20 wherein the sample composition consists of MenB antigens 936-741 (of SEQ ID NO: 1), 287-953 (of SEQ ID NO: 2), 961c (of SEQ ID NO: 3) and OMV (of SEQ ID NO: 4), aluminium hydroxide, sodium chloride, histidine and sucrose.

35. The method according to claim 20 wherein the one or more standard compositions consist of an antigen surrogate, aluminium hydroxide, sodium chloride, histidine and sucrose.

36. The method according to claim 20 wherein the one or more standard compositions do not comprise any proteins other than the antigen surrogate.
